(19) United States
(12) Patent Application Publication
(10) Pub. No.: US 2003/0069271 A1
(43) Pub. Date: Apr. 10, 2003

(54) Quinolone compounds for use in treating viral infections

(76) Inventors: Clarence Webster Andrews,III, Durham, NC (US); George Andrew Freeman, Durham, NC (US); Andrew Lee Hopkins, Neath (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/168,187

(22) PCT Filed: Dec. 15, 2000
(86) PCT No.: PCT/US00/33930

(30) Foreign Application Priority Data
Dec. 20, 1999 (GB) 9930061.8

(51) Int. Cl. A61K 31/4704; A61K 31/4706; C07D 215/16; C07D 215/20; C07D 215/36

(52) U.S. Cl. 514/312; 546/155; 546/157

(57) ABSTRACT

The present invention relates to quinolone compounds and their use in the treatment of viral infections.

### FIELD OF THE INVENTION

The present invention relates to quinolone compounds and their use in medical therapy.

### BACKGROUND OF THE INVENTION

Retroviruses form a sub-group of RNA viruses which, in order to replicate, must first “reverse transcribe” the RNA of their genome into DNA (“transcription” conventionally describes the synthesis of RNA from DNA). Once in the form of DNA, the viral genome may be incorporated into the host cell genome, allowing it to take advantage of the host cell's transcription/translation machinery for the purposes of replication. Once incorporated, the viral DNA is virtually indistinguishable from the host's DNA and, in this state, the virus may persist for the life of the cell.

A species of retrovirus, the Human immunodeficiency virus (HIV) has been reproducibly isolated from patients with AIDS (acquired immunodeficiency syndrome) or with the symptoms that frequently precede AIDS. AIDS is an immunosuppressive or immunodestructive disease that predisposes subjects to fatal opportunistic infections. Characteristically, AIDS is associated with a progressive depletion of T-cells, especially the helper-inducer subset bearing the CD4 surface marker. HIV is cytopathic and appears to preferentially infect and destroy T-cells bearing the CD4 marker, and it is now generally recognized that HIV is the etiological agent of AIDS. Clinical conditions such as AIDS-related complex (ARC), progressive generalized lymphadenopathy (PGL), Karposi's sarcoma, thrombocytopenic purpura, AlDS-related neurological conditions, such as AIDS dementia complex, multiple sclerosis or tropical paraparesis, and also anti-HIV antibody-positive and HIV-positive conditions, including such conditions in asymptomatic patients, are also conditions which may be treated by appropriate anti-viral therapy.

Another RNA virus which has been recognized as the causative agent of an increasingly serious international health problem is the non-A, non-B hepatitis virus. At least 80% of cases of chronic post-transfusional non-A, non-B hepatitis have been shown to be due to the virus now identified as hepatitis C and this virus probably accounts for virtually all cases of

post-transfusional hepatitis in clinical settings where blood products are screened for hepatitis B. Whereas approximately half of the cases of acute hepatitis C infection resolve spontaneously over a period of months, the remainder become chronic and in many if not all such cases chronic active hepatitis ensues with the potential for cirrhosis and hepatocellular carcinoma. The structure of the hepatitis C virus genome has been elucidated and the virus has been characterized as a single stranded RNA virus with similarities to flaviviruses.

The isolation and synthesis of quinolone analogs from the plant family Rutaceae have been described (Indian J. Chemistry 22B, 617-618, 198); Acta Pharmaceutica Sinica 20(4), 277-282, 1985 Tetrahedron 45(3), 757-762,.1989; Liebigs Ann. Chem. 355-358, 1993; Natural Product Letters 3(3), 177-182, 1993; and Tetrahedron 40(18), 3431-3436, 1984).

The synthesis of various halogenated 4-hydroxy-2(1H)-quinolones are described in J. Heterocyclic Chem. 25, 857-862, 1988; Montash. Chem. 123, 617-636, 1992; and Khim. Geterotsikl. Soedin. 2, 204-7, 1995 and the alkylation of 4-hydroxy-2-quinolones has been described in Montash. Chem. 99, 1943-1949, 1968; and Montash. Chem. 99, 1950-1957, 1968.

4-Hydroxy-2-quinolones have been shown to be useful as selective antagonists that can bind at the N-methyl-D-aspartate binding site. These compounds are potential agents for the treatment of central nervous system diseases (EP-481676 A1, European Journal of Pharmacology₂ Molecular Pharmacology Section 290, 221-226, 1995 and Bioorganic and Medicinal Chemistry Letters 3(2), 299-304, 1993).

3-Acyl-2-quinolone derivatives have been shown to be useful in agriculture, as well as potential anti-inflammatory drugs (JP02152966 A2; WO 92/17452; JP-0146192; DE4138820; and Khim. Geterotsikl. Soedin. 10, 1397-1399, 1994).

It has now been discovered that certain quinolone compounds are useful for the treatment of viral infections, particularly retroviral infections, especially HIV.

### SUMMARY OF THE INVENTION

The present invention relates to compounds of formula (Ia)

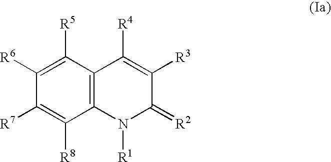

wherein:

R¹ is hydrogen;

R² is oxygen or sulfur;

R³ is trifluoromethyl; cyano; C₁₋₈alkyl optionally substituted with C₁₋₈alkyl or trifluoromethyl;

or OR¹⁵, wherein R¹⁵ is C₁₋₈alkyl optionally substituted with C₁₋₈alkyl;

R⁴ is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocycleaLkyl;

heterocyclealkynyl; C₃₋₆cycloalkylalkenyl; C₆₋₁₄arylamynyl; C₃₋₆cycloalkylalkynyl;

SR¹², wherein R¹² is C₃₋₆cycloalkyl;

S(O)R¹², wherein R¹² is C₃₋₆cycloalkyl; or

NR¹³R¹⁴wherein R¹³ and R¹⁴, which may be the same or different, are hydrogen or C₁₋₈alkyl, optionally substituted with C₁₋₈alkyl;

R⁵ is hydrogen; nitro; halogen; C₁₋₈alkyl, optionally substituted with C₁₋₈alkyl or trifluoromethyl;

R⁶ is hydrogen; halogen; C₁₋₈alkyl; cyano; trifluoromethyl; or OR¹⁰ wherein R¹⁰ is C₁₋₈alkyl or trifluoromethyl;

R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

provided that R⁶ and R⁷ cannot both be hydrogen; and further provided that when R¹ is H, R² is O, R³ is C₁₋₈alkyl, R⁴ is OR¹¹ wherein R¹¹ is C₁₋₈alkyl, R⁵ is H, R⁶ is H or OR¹⁰ wherein R¹⁰ is C₁₋₈alkyl, R⁷ is H, C₁₋₈alkyl, or OR⁹ wherein R⁹ is C₁₋₈alkyl, then R⁸ cannot be H or OR¹⁶ wherein R¹⁶ is H or C₁₋₈alkyl;

or a pharmaceutically acceptable derivative thereof.

The present invention also relates to compounds of formula (Ib)

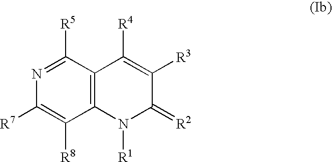

wherein:

R¹ is hydrogen;

R² is oxygen or sulfur;

R³ is trifluoromethyl; cyano; C₁₋₈alkyl optionally substituted with C₁₋₈alkyl or trifluoromethyl;

or OR¹⁵, wherein R¹⁵ is C₁₋₈alkyl optionally substituted with C₁₋₈alkyl;

R⁴ is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;

heterocyclealkynyl; C₃₋₆cycloalkylalkenyl; C₆₋₁₄arylalkynyl; C₃₋₆Cycloalkylalkynyl;

SR¹², wherein R¹² is C₃₋₆Cycloalkyl;

S(O)R¹², wherein R¹² is C₃₋₆cycloalkyl; or

NR¹³R¹⁴ wherein R¹³ and R¹⁴, which may be the same or different, are hydrogen or C₁₋₈alkyl, optionally substituted with C₁₋₈alkyl;

R⁵ is hydrogen; nitro; halogen; C₁₋₈alkyl, optionally substituted with C₁₋₈alkyl or trifluoromethyl;

R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

or a pharmaceutically acceptable derivative thereof.

The present invention also relates to compounds of formula (Ic)

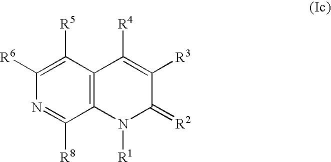

wherein:

R¹ is hydrogen;

R² is oxygen or sulfur;

R³ is trifluoromethyl; cyano; C₁₋₈alkyl optionally substituted with C₁₋₈alkyl or trifluoromethyl;

or OR¹⁵, wherein R¹⁵ is C₁₋₈alkyl optionally substituted with C₁₋₈alkyl;

R⁴is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;

heterocyclealkynyl; C₃₋₆cycloalkylalkenyl; C₆₋₁₄arylalkynyl; C₃₋₆cycloalkylalknyl;

SR¹², wherein R¹² is C₃₋₆cycloalkyl;

S(O)R¹², wherein R¹² is C₃₋₆cycloalkyl; or

NR¹³R¹⁴ wherein R¹³ and R¹⁴, which may be the same or different, are hydrogen or C₁₋₈alkyl, optionally substituted with C₁₋₈alkyl;

R⁵ is hydrogen; nitro; halogen; C₁₋₈alkyl, optionally substituted with C₁₋₈alkyl or trifluoromethyl;

R⁶ is hydrogen; halogen; C₁₋₈alkyl; cyano; trifluoromethyl; or OR¹⁰ wherein R¹⁰ is C₁₋₈alkyl or trifluoromethyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

or a pharmaceutically acceptable derivative thereof

### DETAILED DESCRIPTION OF THE INVENTION

The present invention features compounds of formula (Ia)

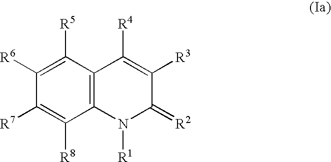

wherein:

R¹ is hydrogen;

R² is oxygen or sulfur; R³ is trifluoromethyl; cyano; C₁₋₈alkyl optionally substituted with C₁₋₈alkyl or trifluoromethyl;

or OR¹⁵, wherein R¹⁵ is C₁₋₈alkyl optionally substituted with C₁₋₈alkyl;

R⁴ is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;

heterocyclealkynyl; C₃₋₆cycloalkylalkenyl; C₆₋₁₄arylalkynyl; C₃₋₆cycloalkylalkynyl;

SR¹², wherein R¹² is C₃₋₆cycloalkyl;

S(O)R¹², wherein R¹² is C₃₋₆cycloalkyl; or

NR¹³R¹⁴ wherein R¹³ and R¹⁴, which may be the same or different, are hydrogen or C₁₋₈alkyl, optionally substituted with C₁₋₈alkyl;

R¹ is hydrogen; nitro; halogen; C₁₋₈alkyl, optionally substituted with C₁₋₈alkyl or trifluoromethyl;

R⁶ is hydrogen; halogen; C₁₋₈alkyl; cyano; trifluoromethyl; or OR¹⁰ wherein R¹⁰ is C₁₋₈alkyl or trifluoromethyl;

R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

provided that R₆ and R₇ cannot both be hydrogen; and further provided that when R¹ is H, R² is o, R³ is C₁₋₈alkyl, R⁴ is OR¹¹ wherein R¹¹ is C₁₋₈alkyl, R⁵ is H, R⁶ is H or OR¹⁰ wherein R¹⁰ is C₁₋₈alkyl, R⁷ is H, C₁₋₈alkyl, or OR⁹ wherein R⁹ is C₁₋₈alkyl, then R⁸ cannot be H or OR¹⁶ wherein R⁶ is H or C₁₋₈alkyl;

or a pharmaceutically acceptable derivative thereof.

The present invention also features compounds of formula (Ib)

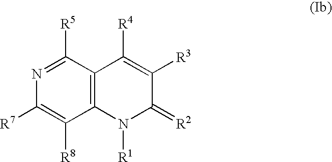

wherein:

R¹ is hydrogen;

R² is oxygen or sulfur;

R³ is trifluoromethyl; cyano; C₁₋₈alkyl optionally substituted with C₁₋₈alkyl or trifluoromethyl;

OR¹⁵, wherein R¹⁵ is C₁₋₈alkyl optionally substituted with C₁₋₈alkyl;

R⁴is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;

heterocyclealkynyl; C₃₋₆cycloalkylalkenyl; C₆₋₁₄arylalkynyl; C₃₋₆cycloalkylalkynyl;

SR¹², wherein R¹² is C₃₋₆cycloalkyl;

S(O)R¹², wherein R¹² is C₃₋₆cycloalkyl; or

NR¹³R¹⁴ wherein R¹³ and R¹⁴, which may be the same or different, are hydrogen or C₁₋₈alkyl, optionally substituted with C₁₋₈alkyl;

R⁵ is hydrogen; nitro; halogen; C₁₋₈alkyl, optionally substituted with C₁₋₈alkyl or trifluoromethyl;

R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

or a pharmaceutically acceptable derivative thereof.

The present invention also features compounds of formula (Ic)

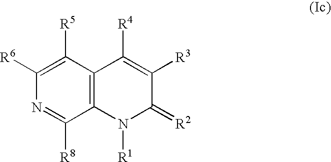

wherein:

R¹ is hydrogen;

R² is oxygen or sulfur;

R³ is trifluoromethyl; cyano; C₁₋₈alkyl optionally substituted with C₁₋₈alkyl or trifluoromethyl;

or OR¹⁵, wherein R¹⁵ is C₁₋₈alkyl optionally substituted with C₁₋₈alkyl;

R⁴is

OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;

heterocyclealkynyl; C₃₋₆cycloalkylalkenyl; C₆₋₁₄arylalkynyl; C₃₋₆cycloalkylalkynyl;

SR¹², wherein R¹² is C₃₋₆cycloalkyl;

S(O)R¹², wherein R¹² is C₃₋₆cycloalkyl; or

NR¹³R¹⁴ wherein R¹³ and R¹⁴, which may be the same or different, are hydrogen or C₁₋₈alkyl, optionally substituted with C₁₋₈alkyl;

R⁵ is hydrogen; nitro; halogen; C₁₋₈alkyl, optionally substituted with C₁₋₈alkyl or trifluoromethyl;

R⁶ is hydrogen; halogen; C₁₋₈alkyl; cyano; trifluoromethyl; or OR¹⁰ wherein R¹⁰ is C₁₋₈alkyl or trifluoromethyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

or a pharmaceutically acceptable derivative thereof.

The term “alkyl” refers to a straight-chain or branched-chain saturated aliphatic hydrocarbon radical containing the specified number of carbon atoms, or where no number is specified, preferably from 1 to about 10, more preferably from 1 to about 8 carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like. The term “alkenyl,” alone or in combination with any other term, refers to a straight-chain or branched-chain alkyl group with at least one carbon-carbon double bond. Examples of alkenyl radicals include, but are not limited to, ethenyl, propenyl, isopropenyl, butenyl, isobutyenyl, pentenyl, hexenyl, hexadienyl and the like.

The term “alknyl” refers to hydrocarbon groups of either a straight or branched configuration with one or more carbon-carbon triple bonds which may occur in any stable point along the chain, such as ethynyl, propynyl, butynyl, pentynyl, and the like.

The term “aryl” refers to a carbocyclic aromatic radical (such as phenyl or naphthyl) containing the specified number of carbon atoms, preferably from 6-14 carbon atoms, and more preferably from 6-10 carbon atoms, optionally substituted with one or more substituents selected from C₁₋₆alkoxy (for example, methoxy), nitro, halogen (for example chloro), amino, carboxylate and hydroxy. Examples of aryl radicals include, but are not limited to phenyl, naphthyl, indenyl, indanyl, azulenyl, fluorenyl, anthracenyl and the like.

The term “heterocycle”, alone or in combination with another term, refers to a stable 3-7 membered monocyclic heterocyclic ring or 8-11 membered bicyclic heterocyclic ring which is either saturated or unsaturated, and which may be optionally benzofused if monocyclic. Each heterocycle consists of one or more carbon atoms and from one to four heteroatoms selected from the group consisting of nitrogen, oxygen and sulfur. As used herein, the terms “nitrogen and sulfur heteroatoms” include any oxidized form of nitrogen and sulfur, and the quaternized form of any basic nitrogen. A heterocyclyl radical may be attached at any endocyclic carbon or heteroatom which results in the creation of a stable structure. Preferred heterocycles include 5-7 membered monocyclic heterocycles and 8-10 membered bicyclic heterocycles. Examples of such groups include imidazolyl, imidazolinoyl, imidazolidinyl, quinolyl, isoqinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetrahydropyranofuranyl.

Preferred heterocycles include imadazolyl, pyrrolyl, pyrrolinyl, piperidinyl, piperazinyl, and morpholinyl.

The term “halogen” refers to a radical of fluorine, chlorine, bromine or iodine.

The term “pharmaceutically acceptable derivative”, as used herein, means any pharmaceutically acceptable salt, ester, salt of an ester, or other derivative of a compound of this invention which, upon administration to a recipient, is capable of providing (directly or indirectly) a compound of this invention or an inhibitorily active metabolite or residue thereof. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species.

Compounds of formula (Ia), (Ib) and (Ic) and their pharmaceutically acceptable derivatives may hereinafter be referred to as compounds of the present invention.

Many quinolone derivatives described in the literature are substituted on the N-1 position and have no substituents in the benzenoid portion of the quinolone. The compounds of the present invention are novel in several respects. We have discovered that the N-1 position of the quinolone must be unsubstituted (that is, R¹ is hydrogen) in order to observe potent anti-HIV activity. Compounds of the present invention that are particularly advantageous are those that are substituted in the benzenoid portion of the quinolone ring system, that is —R⁶ and —R⁷ are not simultaneously hydrogen. We have further discovered that increased antiviral activity occurs when the C-3 position is substituted with alkyl or branched alkyl. In addition, we have discovered that substitution at the 4-position of the quinolone system with —OR¹¹, —SR¹², S(O)₂R¹² or —NR¹³R¹⁴ is required in order to observe anti-HIV activity.

The simultaneous combination of the substitution patterns described above provides compounds that possess surprising anti-viral activity in comparison to 2-quinolones that are described in the literature.

The present invention features compounds of formula (Ia) wherein

R¹ is hydrogen;

R² is oxygen;

R³ is trifluoromethyl; cyano; C₁₋₈alkyl optionally substituted with C₁₋₈alkyl or trifluoromethyl;

R⁴is

OR¹¹, wherein R¹¹ is C₂₋₈alkenyl optionally substituted with C₁₋₈alkyl; C₆₋₁₄arylalkyl; C₃₋₆cycloalkyl; C₃₋₆cycloalklylalkyl; heterocyclealkyl; heterocyclealkynyl; C₃₋₆cycloalkylalkenyl; C₆₋₁₄arylalkynyl; C₃₋₆cycloalkylalkynyl;

SR¹², wherein R¹² is C₃₋₆cycloalkyl; or

S(O)R¹², wherein R¹² is C₃₋₆cycloalkyl;

R⁵ is hydrogen; nitro; halogen; C₁₋₈alkyl, optionally substituted with C₁₋₈alkyl or trifluoromethyl;

R⁶ is halogen; cyano; trifluoromethyl;

R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;

R⁸ is hydrogen; halogen; cyano; nitro; or OR¹⁶, wherein R¹⁶ is hydrogen or C₁₋₈alkyl optionally substituted with C₁₋₈alkyl or trifluoromethyl;

or a pharmaceutically acceptable derivative thereof.

A preferred aspect of the present invention features a compound of formula (Ia) wherein

R¹ is hydrogen;

R² is oxygen;

R³ is C₁₋₈alkyl optionally substituted with C₁₋₈alkyl;

R⁴ is OR¹¹, wherein R¹¹ is C₁₋₈alkyl optionally substituted with C₁₋₈alkyl;

C₆₋₁₄arylalkyl; C₃₋₆cycloalkyl; C₃₋₆cycloalkylalkyl; heterocyclealkyl; C₃₋₆cycloalkylalkyl;

C₃₋₆cycloalkylalkynyl; or SR² wherein R¹² is C₃₋₆cycloalkyl;

R⁵, R⁷, and R⁸ are hydrogen;

R⁶ is halogen;

or a pharmaceutically acceptable derivative thereof.

In another preferred aspect of the present invention there is provided compounds of formula (Ia) wherein:

R¹ is hydrogen;

R² is oxygen;

R³ is C₁₋₈alkyl optionally substituted with C₁₋₈alkyl;

R⁴ is OR¹¹, wherein R¹¹ is C₆₋₁₄arylalkyl, C₃₋₆cycloalkyl, C₃₋₆cycloalkylalkyl, heterocyclealkyl, C₃₋₆cycloalkylalkenyl, or C₃₋₆cycloalkylalkynyl;

R⁵, R⁷,and R⁸are hydrogen;

R⁶ is halogen;

or a pharmaceutically acceptable derivative thereof.

A preferred aspect of the present invention features a compound of formula (Ib) wherein

R¹ is hydrogen;

R² is oxygen;

R³ is C₁₋₈alkyl optionally substituted with C₁₋₈alkyl;

R⁴ is OR¹¹, wherein R¹¹ is C₁₋₈alkyl optionally substituted with C₁₋₈alkyl;

C₆₋₁₄arylalkyl; C₃₋₆cycloalkyl; C₃₋₆cycloalkylalkyl; heterocyclealkyl;C₃₋₆cycloalkylalkenyl;

C₃₋₆cycloalkylalkynyl; or SR¹² wherein R¹² is C₃₋₆cycloalkyl;

R⁵, R⁷, and R⁸ are hydrogen;

or a pharmaceutically acceptable derivative thereof.

A preferred aspect of the present invention features a compound of formula (Ic) wherein

R¹ is hydrogen;

R² is oxygen;

R³ is C₁₋₈alkyl optionally substituted with C₁₋₈alkyl;

R⁴ is OR¹¹, wherein R¹¹ is C₁₋₈alkyl optionally substituted with C₁₋₈alkyl; C₆₋₁₄arylalkyl; C₃₋₆cycloalkyl; C₃₋₆cycloalkylalkyl; heterocyclealkyl; C₃₋₆cycloalkylalkenyl; C₃₋₆cycloalkylalkynyl; or SR¹² wherein R¹² is C₃₋₆cycloalkyl;

R⁵ is hydrogen;

R⁶ is halogen;

R⁸ is hydrogen;

or a pharmaceutically acceptable derivative thereof.

The present invention features a compound selected from the group consisting of

6-Chloro-4-(cyclohexyloxy)-3-propyl-2(1H)-quinolinone;

6-Chloro-4-(cyclobutyhnethoxy)-3-propyl-2(1H)-quinolinone;

6-Chloro-4-(cyclopropylmethoxy)-3-propyl-2(1H)-quinolinone;

6-Chloro-4-(cyclopentyloxy)-3-propyl-2(1H)-quinolinone;

6-Chloro-4-(cyclohexylmethoxy)-3-propyl-2(1H)-quinolinone;

6-Chloro-4-(1 ,3-dioxolan-2-ylmethoxy)-3-propyl-2(1H)-quinolinone;

4Benzyloxy-6-chloro-3-propyl-2(1H)-quinolmone;

6-Chloro-4-(cyclobutyloxy)-3-propyl-2(1H)-quinolinone;

6-Chloro-4-(cyclohexyloxy)-3-isopropyl-2(1H)-quinolinone;

4-Cyclopentyloxy-6-methyl-3-propyl-2(1H)-quinolinone;

4-Cyclopentyloxy-6-methoxy-3-propyl-2(1H)-quinolinone;

4-(Cyclopentyloxy)-3-propyl-6-trifluoromethoxy-2(1H)-quinolinone;

4-Cyclopentyloxy-2-oxo-3-propyl-1 ,2-dihydro-6-quinolinecarbonitrile

6-Bromo-4-(cyclopentyloxy)-3-propyl-2(1H)-quinolinone;

4Cyclopentyloxy-3-propyl-2(1H)-quinolinone;

6-Chloro-4-cyclobutylmethoxy-3-isopropyl-2(1H)-quinolinone;

6-Chloro-4-(cyclopentyloxy)-3-ethyl-2(1H)-quinolinone;

3-(sec-Butyl)-6-chloro-4-(cyclopentyloxy)-2(1H)-quinolinone;

6-Chloro-4-(cyclopentyloxy)-3-isobutyl-2(1H)-quinolinone;

6-Chloro-4-(cyclohexyloxy)-3-propyl-2(1H)-quinolinethione;

6-Chloro-4-(cyclobutylmethoxy)-3-propyl-2(1H)-quinolinethione;

3-(sec-Butyl)-6-chloro-4-(cyclopentyloxy)-2(1H)-quinolinethione;

6-Chloro-4-(cyclohexylsulfanyl)-3-propyl-2(1H)-quinolinone;

6-Chloro-4-(cyclohexylsulfinyl)-3-propyl-2(1H)-quinolinone;

4-Cyclopentyloxy-6-fluoro-3-propyl-2(1H)-quinolinone;

4-Cyclobutylmethoxy-6-fluoro-3-propyl-2(1H)-quinolinone;

3-sec-Butyl-4-(cyclobutylmethoxy)-6-fluoro-2(1H)-quinolinone;

4-(Cyclobutylmethoxy)-6-fluoro-3-isopropyl-2(1H)-quinolinone;

4-Cyclopentyloxy-6-fluoro-3-propyl-2(1H)-quinolinethione;

4-(Cyclobutylmethoxy)-6-fluoro-3-propyl-2(1H)-quinolinethione;

4-(Cyclobutylmethoxy)-6-fluoro-3-isopropyl-2(1H)-quinolinethione;

6-Chloro-4-(isobutylamino)-3-propyl-2(1H)-quinolinone;

6- Chloro4-(cyclobutylmethoxy)-3-ethoxy-2(1H)-quinolinone;

4-(Cyclobutylmethoxy)-3-ethoxy-6-fluoro-2(1H)-quinolinone;

6-Chloro-4-[(2-cyclopropylethynyl)oxy]-3-propyl-2(1H)-quinolinone;

4-[(2-Cyclopropylethynyl)oxy]-6-fluoro-3-(isopropyl)-2(1H)-quinolinone;

4-[(2-Cyclopropylethynyl)oxy]-6-fluoro-3-propyl-2(1H)-quinolinone;

6-Fluoro-3-isopropyl-4-[(3-methyl-1-pentynyl)oxy]-2(1H)-quinolinone;

4-(Cyclobutylmethoxy)-3-ethyl-6-fluoro-2(1H)-quinolinone;

4-Cyclobutylmethoxy-3-isopropyl[1,6]naphthyridin-2(1H)-one;

3-sec-Butyl-4-cyclobutylmethoxy[1 ,6]naphthyridin-2(1H)-one;

6-Fluoro-3-isopropyl-4-[(3-methyl-2-butenyl)oxyl-2(1H)-quinolinone;

6-Fluoro-3-isopropyl-4-[(2-methyl-2-propenyl)oxy]-2(1H)-quinolinone;

4-(Cyclobutylmethoxy)-6-fluoro-5-nitro-3-propyl-2(1H)-quinolinone;

4-[(2-Cyclopropylethynyl)oxy]-3-ethyl-6-fluoro-2(1H)-quinolinone;

6-Chloro-4-[(2-Cyclopropylethynyl)oxy]-3-isopropyl-2(1H)-quinolinone;

4-[(2-Cyclopropylethynyl)oxy]-6-fluoro-3-isobutyl-2(1H)-quinolinone;

4-{[(E)-2-Cyclopropylethenyl]oxy}-6-fluoro-3-isopropyl-3,4-dihydro-2(1H)-quinolinone;

4-Cyclobutylmethoxy-6-fluoro-3-methyl-2(1H)-quinolinone;

and pharmaceutically acceptable derivatives thereof.

Preferred compounds of the present invention are:

6-Chloro-4-[(2-cyclopropylethynyl)oxy]-3-propyl-2(1H)-quinolinone (example 35),

4-[(2-Cyclopropylethynyl)oxy]-6-fluoro-3-propyl-2(1H)-quinolinone (example 37),

4-[(2-Cyclopropylethynyl)oxy]-3-ethyl-6-fluoro-2(1H)-quinolinone (example 45),

6-Chloro-4-[(2-Cyclopropylethynyl)oxy]-3-isopropyl-2(1H)-quinolinone (example 46),

4-[(2-Cyclopropylethynyl)oxy]-6-fluoro-3-isobutyl-2(1H)-quinolinone (example 47),

4-[(2-Cyclopropylethynyl)oxy]-6-fluoro-3-isopropyl-2(1H)-quinolinone (example 36 and

pharmaceutically acceptable derivatives thereof.

Preferred esters in accordance with the invention may include (1) carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄ alky, or C₁₋₄ alkoxy), or amino; (2) sulphonate esters, such as alkyl- or aralkylsulphonyl (for example, methanesulphonyl); (3) amino acid esters (for example, L-valyl or L-isoleucyl); and (4) phosphonate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group. Any reference to any of the above compounds also includes a reference to a physiologically acceptable salt thereof.

Examples of physiologically acceptable salts of compounds according to the invention include salts derived from an appropriate base, such as an alkali metal (for example, sodium), an alkaline earth (for example, magnesium), ammonium and NX₄⁺ (wherein X is C₁₋₄ alkyl).

Physiologically acceptable salts of an hydrogen atom or an amino group include salts of organic carboxylic acids such as acetic, lactic, tartaric, malic, isethionic, lactobionic, and succinic acids, organic sulphonic acids, such as methanesulphonic, ethanesulphonic, benzenesulphonic and p-toluenesulphonic acids and inorganic acids, such as hydrochloric, sulphuric, phosphoric and sulphamic acids. Physiologically acceptable salts of compounds according to the invention with an hydroxy group include the anion of said compound in combination with a suitable cation such as Na⁺, NH₄⁺ and NX₄⁺ (wherein X is a C₁₋₄ alkyl group).

For therapeutic use, salts of compounds according to the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived from a physiologically acceptable acid or base, are within the scope of the present invention.

The present invention further includes a process for the preparation of a compound according to the invention, which comprises compounds for formulas (Ia), (Ib), and (Ic).

The novel compounds contained in this invention can prepared according to Schemes I-VI, which are presented below. The compounds, which can be prepared according to these schemes, are not limited by the compounds contained in the schemes or by any particular substituents used in the schemes for illustrative purposes. The examples contained in this invention specifically illustrate the application of the following schemes to specific compounds.

Scheme I, below, is a general route for synthesizing compounds of general formula (Ia) in which R¹, R⁵, R⁷, and R⁸ are hydrogen, R⁶ is halogen, R² is oxygen or sulfur, R³ is C₁₋₈alkyl optionally substituted with C₁₋₈alkyl or trifluoromethyl, R⁴ is —OR¹¹, where R¹¹ is C₆₋₁₄arylalkyl, C₃₋₆cycloalkyl, C₃₋₆cycloalkylalkyl, or heterocyclealkyl. As shown in Scheme I, 4-substituted anilines (1), which are commercially available or are readily prepared by those skilled in the art, are treated with an excess of a substituted malonate ester. The malonate esters are commercially available or are readily prepared by those skilled in the art. This reaction is conducted in a high-boiling, aprotic solvent, such as diphenyl ether, at an elevated temperature, preferably 250° C., for 24 hours. The resulting quinolone product (2) is then allowed to react with an alkylating agent that contains a suitable leaving group in the presence of a base in a polar solvent and at an elevated temperature. The alkylating agent must contain a leaving group such as a bromide, iodide, para-toluenemethanesulfonate ester, or trifluoromethanesulfonate ester, in order to assure that it is reactive enough to alkylate the hydroxy group of the intermediate quinolone (2). The base can be either inorganic, preferably potassium carbonate, or organic, diisopropylethylamine for example or preferably triethylamine. Polar, high-boiling, aprotic solvents are preferred, N,N-dimethylformanmide for instance. An elevated temperature is required for the alkylation reaction, 100° C. for instance.

Alternatively, the intermediate quinolone (2) can be reacted with a stronger base such that deprotonation of the hydroxy group occurs, followed by reaction with a suitable alkylating agent. For example, quinolones of formula I can be allowed to react with a strong base, sodium hydride for example, followed by reaction with an alkylating agent, for example an alkyl bromide. These reactions are preferably conducted in a polar, aprotic solvent such as N,N-dimethylformamide in a temperature range of room temperature-120° C., preferably 50-100° C.

Quinolones of general formula (Ia) wherein R² is sulfur, R¹, R⁵, R⁷, and R⁸ are hydrogen, R⁶ is halogen, R³ is C₁₋₈alkyl optionally substituted with C₁₋₈alkyl or trifluoromethyl, and R⁴ is —OR¹¹, where R¹¹ is C₆₋₁₄arylalkyl, C₃₋₆cycloalkyl, C₃₋₆cycloalkylalkyl, or heterocycleal prepared from quinolones of formula (Ia) in which R² is oxygen and R¹, R⁵, R⁷, and R⁸ are hydrogen, R⁶ is halogen, R³ is C₁₋₈alkyl optionally substituted with C₁₋₈alkyl or trifluoromethyl, and R⁴ is —OR¹¹, where R¹¹ is C₆₋₁₄arylalkyl, C₃₋₆cycloalkyl, C₃₋₆cycloalkylalkyl, or heterocyclealkyl, by reaction with a suitable sulfurizing agent in a high-boiling, aprotic solvent and at an elevated temperature. For example, reaction of quinolone intermediates (3) can be allowed to react with Lawesson's reagent (2,4-bis[4-methoxyphenyl]-1,3-dithia-2,4-diphosphetane-2,4-disulfide) in a solvent such as toluene at an elevated temperature, for example 100° C., for about one hour.

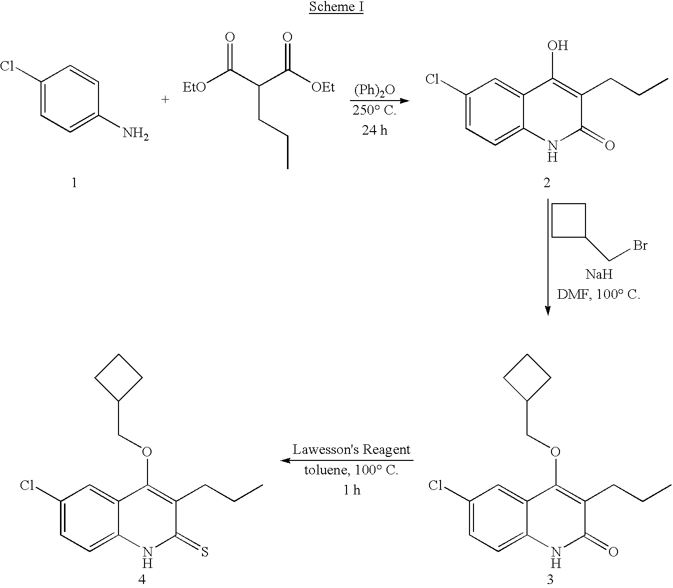

As shown in Scheme II, quinolones of formula (Ia), wherein R¹, R⁵, R⁷ and R⁸ are hydrogen, R² is oxygen, R⁴ is —SR¹², wherein R¹² is C₃₋₆cycloalkyl, and R³ and R⁶ are as hereinbefore defined, can be prepared from quinolones such as (2). Reaction of a quinolone such as (2) with a suitable dehydrating/brominating agent followed by reaction with water in the presence of an acid catalyst and at an elevated temperature provides 4-bromoquinolones such as (6). Reaction of bromoquinolone (6) with a sulfide salt in a polar, aprotic solvent and at ambient temperature provides 4-thioquinolones such as (7). The 4-thioquinolones such as (7) can be oxidized in the presence of a suitable oxidizing agent in a suitable solvent to provide quinolones such as (7) containing a sulfoxide at the 4-position, like compound (8).

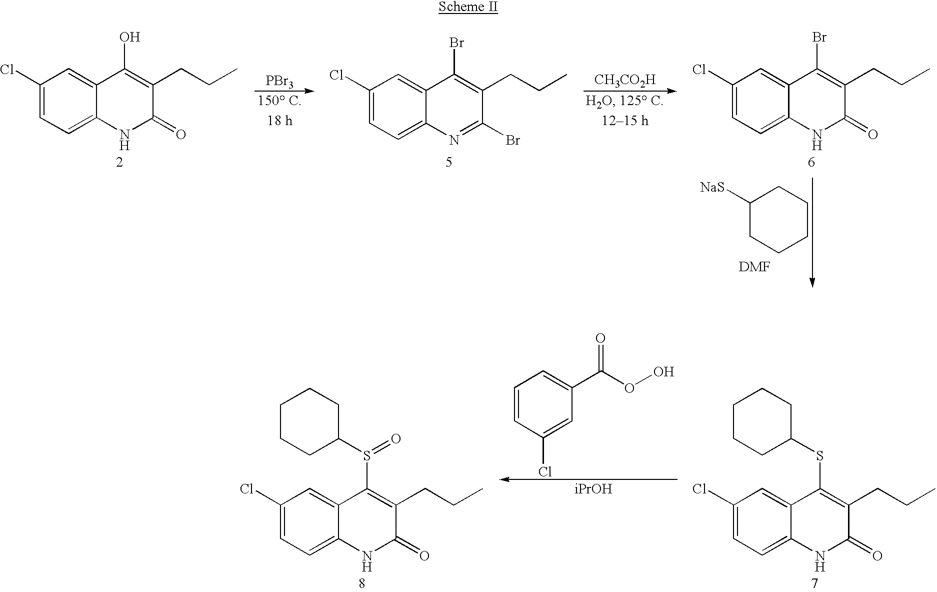

For example, reaction of compound (Ia), where R⁴ is hydrogen and R¹, R², R³, R⁵, R⁶, R⁷ and R⁸ are as hereinbefore defined, may be allowed to react with a dehydrating and brominating agent, such as phosphorous tribromide, at 150° C. for 18 hours to provide dibromide (5). The dibromide is then allowed to react with water in the presence of acetic acid at 125° C. for 12 to 15 hours to provide a 4bromoquinolone such as compound (6). The 4-bromoquinolone (6) can the be reacted with the salt of a suitable alkylthio compound, such as sodium cyclohexylthiol, in N,N-dimethylformamide to provide the corresponding 4-alkylthioether derivative (7). The alkylthio salts of interest can be readily prepared by those skilled in the art from the corresponding thiols by reaction with an appropriate base, for example sodium hydride. The thiols of interest are either commercially available or are readily obtained by methods familiar to those skilled in the art. Oxidation of the 4-alkylthioethers (7) can be readily accomplished by reaction with a suitable oxidizing agent, meta-chloroperbenzoic acid for example, to provide the desired 4-alkylsulfoxide derivative (8).

As shown in Scheme III, compounds of formula (Ia) in which R⁴ is —NR¹³R¹⁴, where R¹³ and R¹⁴ are as hereinbefore defined, and R¹, R², R³, R⁵, R⁶, R⁷ and R⁸ are as hereinbefore defined, can be prepared from compounds of formula (Ia) in which R⁴ is a halogen, bromine for example, by reaction with a suitable primary or secondary amine, in the presence of a base and in a polar, aprotic solvent at an elevated temperature.

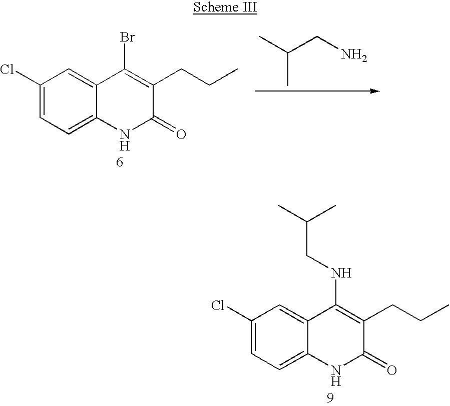

For example, a 4bromoquinolone such as (6) may be allowed to react with either a primary or secondary amine in ethanol at 140° C. to provide the corresponding 4-aminoquinolone. The primary or secondary amines of interest can be obtained commercially or are readily available to those skilled in the art.

As shown in Scheme IV, compounds of formula (Ia) in which R¹, R², R⁴, R⁵, R⁶, R⁷ and R⁸ are as hereinbefore defined and R³ is —OR¹¹, where R¹¹ is as hereinbefore defined, can be prepared from an appropriate anthranilic acid derivative such as (10). Reaction of acid (10) with an alcohol in the presence of a catalytic amount of an acid, in a high-boiling solvent and at an elevated temperature yields the corresponding ester (11). The ester is then allowed to react with the acid chloride of ethoxy acetic acid in the presence of an appropriate base and in an aprotic solvent to yield the corresponding amide (12). The amide (12) is then allowed to react with an excess of a strong base capable of cyclizing the amide (12) to provide the desired quinolone (13). This reaction is best performed in an aprotic solvent and at low temperatures. Alkylation of the 4-hydroxy group in quinolone (13) can be accomplished with a suitable aklylating agent as discussed in relation to Scheme I to provide the desired quinolone (14).

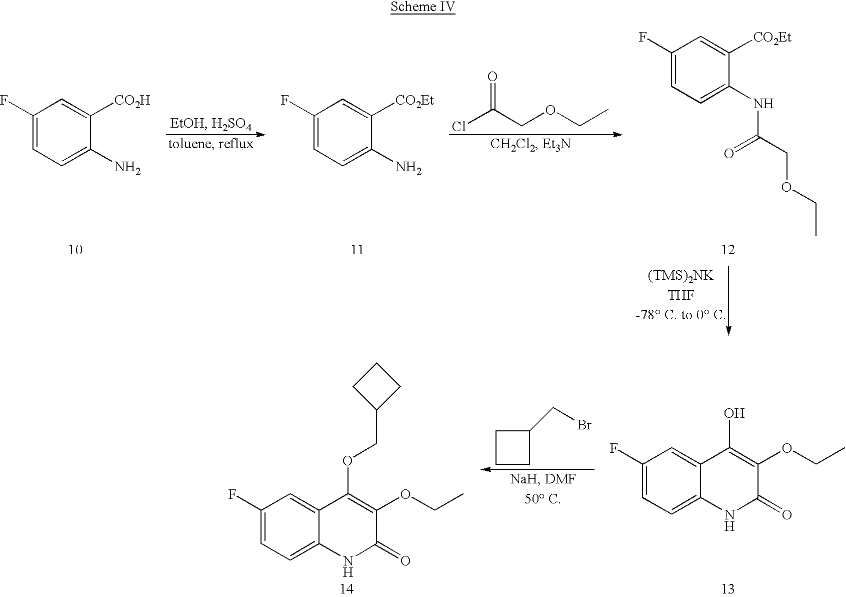

For example, reaction of 2-amino-5-fluorobenzoic acid with an alcohol, such as ethyl alcohol, in the presence of an acid catalyst, sulfuric acid for example, and in a high-boiling solvent such as toluene at an elevated temperature yields the corresponding ethyl ester (11). Anthrinilic acids of interest are readily available commercially or can be easily prepared by those skilled in the art. Reaction of ester (11) with the acid chloride derived from ethoxy acetic acid in the presence of a suitable base, triethylamine for example, and in an aprotic solvent such as dichloromethane, provides the desired amide (12). The acid chloride of ethoxy acetic acid can be readily prepared by those skilled in the art by reaction of the commercially available acid with a chlorinating agent such as thionyl chloride, followed by purification of the acid chloride by distillation. Reaction of amide (12) with at least a three-fold excess of a strong base, such as potassium bis(trimethylsilyl)amide in an aprotic solvent, tetrahydrofuran for example, and at low temperatures, preferably −78° C. to 0° C., provides the desired quinolone (13). Finally, reaction of quinolone (13) with a suitable alkylating agent, such as bromomethylcyclobutane, in the presence of a strong base, sodium hydride for example, and in a polar, aprotic solvent such as N,N-dimethylformamide at 50° C. provides the desired quinolone (14).

As shown in Scheme V, compounds of Formula (Ib), in which R¹, R², R³, R⁵, R⁷ and R⁸ are as hereinbefore defined and R⁴ is —OR¹¹, where R¹¹ is as hereinbefore defined, can be prepared from ethyl 4-aminonicotinate (15), prepared by the method of Ismail and Wibberley, JCS, 1967, p 2613. Reaction of (15) with an appropriate acid chloride in an aprotic solvent and in the presence of a base provides the corresponding amide (16). Acid chlorides of interest are either commercially available or can be readily prepared by those skilled in the art. Cyclization of (16) is accomplished by reaction with a strong base in an aprotic solvent to furnish (17). Quinolone (17) can then be alkylated as described for similar compounds in Scheme I to afford the desired product (18).

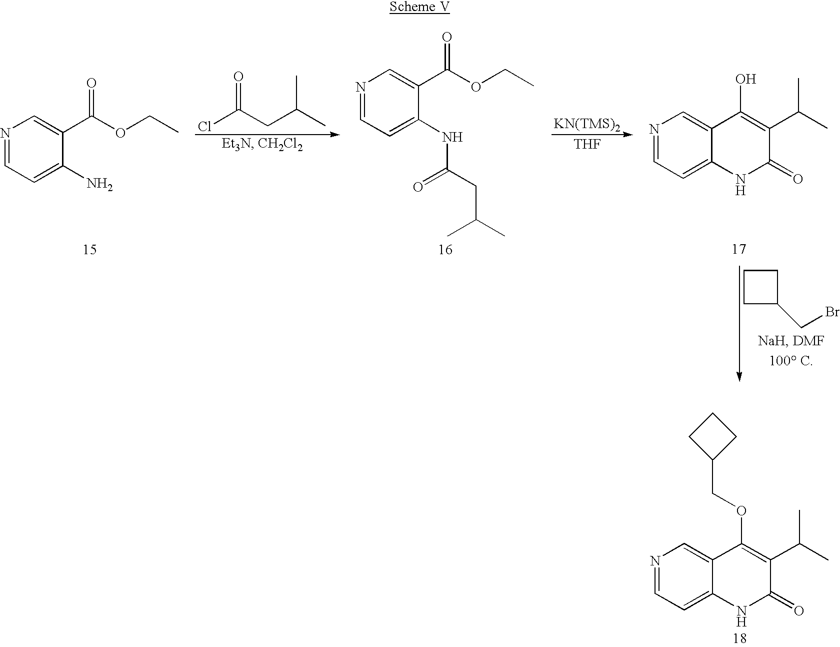

As shown in Scheme VI, compounds of general formula (Ia), in which R¹, R², R³, R⁵, R⁶, R⁷ and R⁸ are as hereinbefore defined, and R⁴ is —OR¹¹, wherein R¹¹ is C₃₋₆cycloalkylalkynyl, can be prepared beginning with a suitable quinolone such as (2). Reaction of quinolone (2) with 1-bromo-2,2,2-trifluoroethane in the presence of a strong base, in a polar, aprotic solvent and at an elevated temperature provides the desired 2,2,2-trifluoroethoxy derivative (19). Protection of the amide (19) can be accomplished with a variety of groups, such as para-methoxybenzyl chloride or tert-butyldimethylsilyl chloride in the presence of a base and in a polar, aprotic solvent to provide protected quinolone (20). The desired alkylalkynyl group is subsequently introduced by allowing (20) to react with an excess of an alkyl lithium reagent in an aprotic solvent and at low temperature to provide intermediate (21).

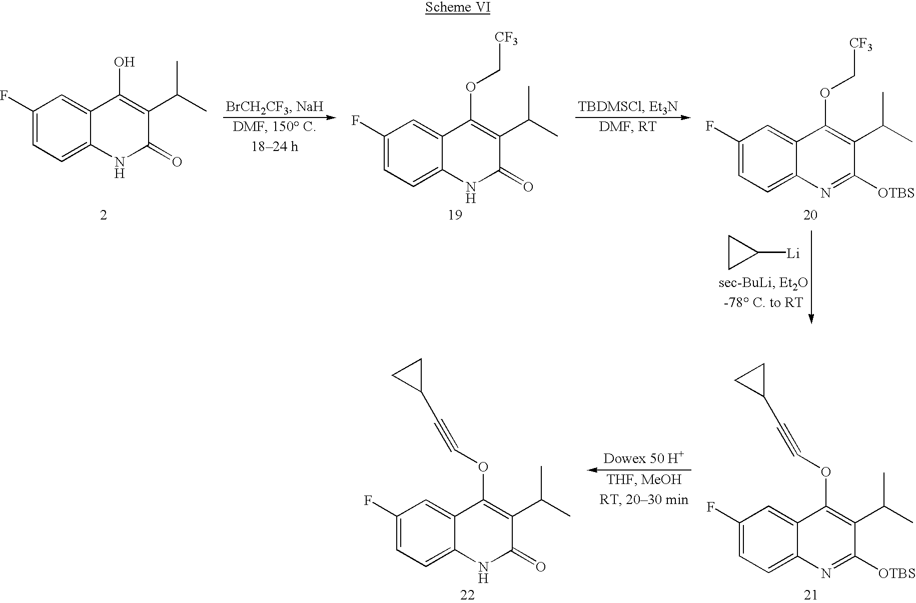

The appropriate alkyl lithium reagents are either commercially available or can be readily prepared by those skilled in the art. Finally, the protecting group can be removed from quinolone (21) using reagents that have been found to remove a particular group under mild conditions and in high yield to provide the desired quinolone (22).

For example, quinolone (2) was allowed to react with 1-bromo-2,2,2-trifluoroethane in the presence of sodium hydride and in N,N-dimethylformamide at 150° C. to provide 2,2,2-trifluoroethoxy derivative (19). Protection of (19) was accomplished by reaction with tert-butyldimethylsilylchloride in N,N-dimethyl formamide and in the presence of triethylamine at ambient temperature to provide silyl derivative (20). Reaction of (20) with cyclopropyllithium, which was pre-formed by reaction of cyclopropylbromide with lithium wire, and sec-butyl lithium in ether at —78° C. followed by warming to room temperature provided alkyne (21). Deprotection of (21) was accomplished by reaction with Dowex 50 acidic resin in a mixture of methanol and tetrahydrofuran at room temperature for 20 to 30 minutes provided the desired quinolone product (22).

As shown in Scheme VII, compounds of formula (Ia) in which R⁴ is —OR¹¹, wherein R¹¹ is C₂₋₆alkenyl and R¹, R², R³, R⁵, R⁶, R⁷ and RW are as hereinbefore defined, can be prepared from compounds such as intermediate 21 shown in Scheme VI. Reaction of protected quinolone 21 with a metal hydride reagent, such as lithium aluminum hydride, in an aprotic solvent, tetrahydrofuran for example, at ambient temperature, followed by treatment of the reaction mixture with acid, provides compounds such 24.

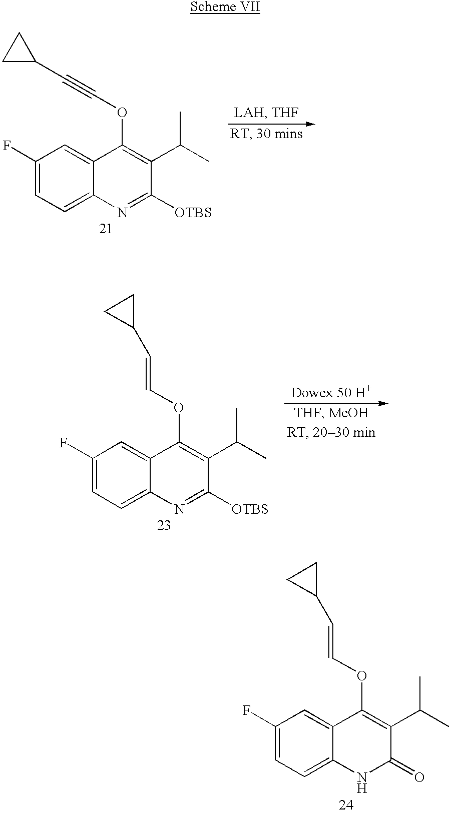

For example, intermediate (21) was allowed to react with lithium aluminum hydride in tetrahydrofuran at room temperature for 30 minutes, to produce intermediate (23). The tert-butyldimethylsilyl protecting group in intermediate (23) was then cleaved under mildly acidic conditions using Dowex 50 acidic resin in a mixture of tetrahydrofuran and methanol at room temperature to afford the desired alkene product (24).

In one aspect of the invention there are provided the compounds according to the invention for use in medical therapy, particularly for the treatment of retroviral infections.

Examples of retroviral infections which may be treated or prevented in accordance with the invention include human retroviral infections such as human immunodeficiency virus HV), HIV-1, HIV-2 and human T-cell lymphotropic virus (HTLV), for example, HTLV-I or HTLV-II infections. The compounds according to the invention are especially useful for the treatment of AIDS and related clinical conditions such as AIDS-related complex (ARC), progressive generalized lymphadenopathy (PGL), Karposi's sarcoma, thrombocytopenic purpura, AIDS-related neurological conditions, such as AIDS dementia complex, multiple sclerosis or tropical paraparesis, anti-HIV antibody-positive and HIV-positive conditions.

The compounds according to the invention are particularly applicable for the treatment of asymptomatic infections or diseases in humans caused by or associated with human retroviruses.

Another aspect of the present invention features the use of a compound according to the invention in the manufacture of a medicament for the treatment of any of the above-mentioned infections or conditions.

A further aspect of the present invention features a method of treatment of a viral infection, particularly an HIV infection, by administration of a compound of the present invention. Any of the above-mentioned infections or conditions may be treated with a compound of the present invention.

The compounds according to the invention may be employed in combination with other therapeutic agents for the treatment of the above infections or conditions. other therapeutic agents may include agents that are effective for the treatment of viral infections or associated conditions such as nucleoside reverse transcriptase inhibitors, for example, zidovudine or abacavir, 2′,3′-dideoxycytidine, 2′,3′-dideoxyadenosine, 2′,3′-dideoxyinosine, 3′-deoxy-2′,3′-didehydrothymidine (d4T); (1 alpha, 2 beta, 3 alpha)-9-[2,3-bis(hydroxymethyl)cyclobutyl]guanine [(−)BHCG, SQ-34514]; oxetanocin-G (3,4-bis(hydroxymethyl)-2-oxetanosyl]guanine); acyclic nucleosides (e.g. acyclovir, valaciclovir, famciclovir, ganciclovir, penciclovir); acyclic nucleoside phosphonates (e.g. (S)-1-(3-hydroxy-2-phosphonyl-methoxypropyl)cytosine (HPMPC), PMEA, PMPA; ribonucleotide reductase inhibitors such as hydroxyurea, protease inhibitors such as indinavir, ritonavir, nelfinavir, amprenavir, sanquinavir; oxathiolane nucleoside analogues such as lamivudine, cis-1-(2-(hydroxymethyl)-1,3-oxathiolan-5-yl)-5-fluorocytosine (FTC), ribavirin, 9-[4-hydroxy-2-(hydroxymethyl)but-1-yl]-guanine (12G), dioxolane G, L-FMAU; tat inhibitors such as 7-chloro-5-(2-pyrryl)-3H-1,4-benzodiazepin-2-(Qone (Ro5-3335), 7-chloro-1,3-dihydro-5-(1H-pyrrol-2yl)-3H-1,4-benzodiazepin-2-amine (Ro24-7429); interferons such as α-interferon; renal excretion inhibitors such as probenecid, nucleoside transport inhibitors such as dipyridamole; pentoxifylline, N-acetylcysteine (NAC), Procysteine, α-trichosanthin, phosphonoformic acid, as well as irnmunomodulators such as interleukin II or thymosin, granulocyte macrophage colony stimulating factors, erythropoetin, soluble CD₄ and genetically engineered derivatives thereof; or non-nucleoside reverse transcriptase inhibitors (NNRTIs) such as nevirapine (13I-RG-587), loviride (α-APA) and delavuridine (BHAP), trovirdine, MKC-442, L737126, and phosphonoformic acid, and 1,4-dihydro-2H-3,1-benzoxazin-2-ones NNRTIs such as efavirnez (DMP-266), and quinoxaline NNRTIs such as isopropyl (2S)-7-fluoro-3,4-dihydro-2-ethyl-3-oxo-1(2H)-quinoxalinecarboxylate (HBY1293). The component compounds of such combination therapy may be administered simultaneously, in either separate or combined formulations, or at different times, for example, sequentially such that a combined effect is achieved. Combination therapies according to the present invention comprise the administration of at least one compound of the formula (Ia), (Ib) or (Ic) or a pharmaceutically acceptable derivative thereof and at least one other pharmaceutically active ingredient. The active ingredient(s) and pharmaceutically active agents may be administered simultaneously in either the same or different pharmaceutical formulations or sequentially in any order. The amounts of the active ingredient(s) and pharmaceutically active agent(s) and the relative timings of administration will be selected in order to achieve the desired combined therapeutic effect. Preferably the combination therapy involves the administration of one compound according to the invention and one of the agents mentioned herein above.

The compounds according to the invention, also referred to herein as the active ingredient, may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the infection and the chosen active ingredient.

The amounts required of the active ingredient will depend upon a number of factors including the severity of the condition to be treated and the identity of the recipient and will ultimately be at the discretion of the attendant physician or veterinarian. In general however, for each of these utilities and indications, a suitable effective dose of a compound of formula (Ia), (Ib) or (Ic) will be in the range of 0.01 to 200 mg per kilogram body weight of recipient per day, advantageously in the range of 1 to 10 mg per kilogram body weight per day.

The desired dose is preferably presented as one, two, three or four or more subdoses administered at appropriate intervals throughout the day. These sub-doses may be administered in unit dosage forms, for example, containing about 25 to 2000 mg, preferably about 25, 50, 150, 200, or 250 mg of active ingredient per unit dose form. While it is possible for the active ingredient to be administered alone, it is preferable to present it as a pharmaceutical composition. A further aspect of the present invention features pharmaceutical compositions comprising a compound of formula (Ia), (Ib) or (Ic) or a pharmaceutically acceptable derivative thereof and a pharmaceutically acceptable carrier therefor.

The compositions of the present invention comprise at least one active ingredient, as defined above, together with one or more pharmaceutically acceptable carriers thereof and optionally other therapeutic agents. Each carrier must be “acceptable” in the sense of being compatible with the other ingredients of the composition and not deleterious to the recipient thereof. Compositions include those suitable for oral, rectal, nasal, topical (including buccal and sublingual), vaginal or parenteral (including subcutaneous, intramuscular, intravenous and intradermal) administration.

The compositions may conveniently be presented in unit dosage form prepared by any of the methods well known in the art of pharmacy. Such methods include the step of bringing into association the active ingredient with the carrier, which constitutes one or more accessory ingredients. In general, the compositions are prepared by uniformly and intimately bringing in to association the active ingredient with liquid carriers or finely divided solid carriers or both, and then, if necessary, shaping the product.

Compositions of the present invention suitable for oral administration may be presented as discrete units such as capsules, cachets, sachets of granules or tablets (such as a swallowable, dispersible or chewable tablet) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compound moistened with an inert liquid diluent. The tablets may optionally be coated or scored any may be formulated so as to provide slow or controlled release of the active ingredient therein.

Compositions suitable for topical administration in the mouth include lozenges comprising the active ingredient in a flavored basis, usually sucrose and acacia or tragacanthl; pastilles comprising the active ingredient in an inert basis such as gelatin and glycerin, or sucrose and acacia; and mouthwashes comprising the active ingredient in a suitable liquid carrier.

Compositions for rectal administration may be presented as a suppository with a suitable base comprising for example cocoa butter or a salicylate.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solution which may contain anti-oxidants, buffers, bacteriostats and solutes which render the formulation aqueous and non-aqueous sterile suspensions which may include suspending agents and thickening agents. The compositions may be presented in unit-dose or multidose sealed containers, for example, ampoules and vial, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The active ingredient may also be presented in a composition comprising micrometer- or nanometer-size particles of active ingredient.

Preferred unit dosage compositions are those containing a daily dose or unit daily sub-dose (as herein above recited) or an appropriate fraction thereof, of the active ingredient.

It should be understood that in addition to the ingredients particularly mentioned above the composition of this invention may include other agents conventional in the art having regard to the type of formulation in question, for example those suitable for oral administration may include flavoring agents or taste masking agents.

A further aspect of the invention relates to kits to be used in the treatment of patients suffering from viral infections. These kits include one or more oral dosage of a compound of formula (I) and may include one or more additional therapeutic agents. By way of illustration, a kit of the invention may include one or more tablets, capsules, caplets, gelcaps or liquid formulations containing a compound of formula (Ia), (Ib), or (ic) and one or more tablets, capsules, caplets, gelcaps or liquid formulations containing a compound of formula (Ia), (Ib) or (ic) in dosage amounts within the ranges described above. The kits may include as an insert printed dosing information for the co-administration of the agents.

The following examples are intended for illustration only and are not intended to limit the scope of the invention in any way.

### EXAMPLE 1

### 6-Chloro-4-(cyclohexyloxy)-3-propyl-2(1H)-quinolinone

A. 6-Chloro-4-hydroxy -3-propyl-2(1H)-quinolinone

4-chloroaniline (10 g, 80 mmol) and diethyl propylmalonate (16 mL, 80 mmol) were combined in phenyl ether (100 mL) and heated to reflux overnight. The reaction was cooled to 50° C. A precipitate formed and was collected by filtration. The product was washed with hexane and used without further purification. The yield was 84%. MS (ES+): m/z 238.0 (M+1); ¹H NMR (DMSO-d₆) δ 11.3 (bs, 1H, OH), 10.0 (bs, 1H, NH), 7.82 (s, 1H, ArH), 7.44 (d, 1H, ArH), 7.21 (d, 1H, ArH), 2.5 (m, overlapping with DMSO), 1.40 (m, 2H, CH₂), 0.86 (t, 3H, CH₃).

B. 6-Chloro-4-(cyclohexyloxy)-3-propyl-2(1H)-quinolinone

6-Chloro-4-hydroxy -3-propyl-2-(1H)-quinolinone (2.50 g, 10.5 mmol), cyclohexyl bromide (2.6 mL, 21 mmol), potassium carbonate (1.6 g, 46 mmol), and triethylamine (1.05 mL, 14 mmol) were combined in DMF (Aldrich, Sure Seal, 50 mL) and heated in a 165° C. oil bath for 36 h. The reaction was poured into ice water (200 mL). The product was extracted with ethyl acetate (1 vol.), dried with magnesium sulfate and filtered. The crude product was purified by chromatography on silica gel eluted with hexane/ethyl acetate (3:1, v/v). A 1% yield was obtained. MS (ES+): m/z 320.0 (M+1); ¹H NMR (DMSO-d₆) δ 11.8 (bs, 1H, NH), 7.62 (s, 1H, ArH), 7.49 (d, 1H, ArH), 7.28 (d, 1H, ArH), 3.95 (m, 1H, CH), 1.95 (m, 2H, CH₂), 1.70 (m, 2H, CH₂), 1.50 (m, 2H, CH₂), 1.28-1.20 (m, 10H, alkyls).

### EXAMPLE 2

### 6-Chloro-4-(cyclobutylmethoxy)-3-propyl-2(1H)-quinolinone

6-Chloro-4-hydroxy-3-propyl-2(1H)-quinolinone (0.50 g, 2.1 mmol) was dissolved in anhydrous DMF (7 mL). Sodium hydride (60% oil dispersion, 0.088 g, 2.2 mmol) was added and the reaction stirred at RT for 10 min. Cyclobutylmethyl bromide (1.1 mL, 11 mmol) was added. The reaction was heated in a 50° C. oil bath for 42 h. The reaction was poured into 50 mL ice water. The product was extracted with ethyl acetate (1 vol). The ethyl acetate solution was dried with magnesium sulfate, filtered, and the solvent removed in vacuo to give a solid. The product was recrystallized from ethyl acetate to give a white solid in 27% yield. MS (APCI−): m/z 304.0 (M−H); ¹H NMR (CDCl₃) δ 12.0 (bs, 1H, NH), 7.67 (s, 1H, ArH), 7.40 (d, 1H, ArH), 7.26 (d, 1H, ArH), 3.97 (d, 2H, OCH₂), 2.88 (m, 1H, CH), 2.66 (m, 2H, alkyls), 2.22 (m, 2H, alkyls), 1.95 (m, 4H, alkyls), 1.65 (m, 2H, CH₂), 1.01 (t, 3H, CH₃).

### EXAMPLE 3

### 6-Chloro-4-(cyclopropylmethoxy)-3-propyl-2(1H)-quinolinone

The title compound was prepared from 6-chloro-4-hydrox-3-propyl-2(1H)-quinolinone by the method used in Example 2 except the reaction was heated in a 100° C. oil bath for 2 h. The crude product was isolated as before. The product was purified by chromatography on silica gel (4×7 cm column) eluted with hexane/ethyl acetate (2:1, v/v) followed by recrystallization from ethyl acetate. A 30% yield was obtained. MS APCI−): m/z 290.0 (M−H); ¹H NMR (DMSO-d₆) δ 11.8 (bs, 1H, NH), 7.67 (s, 1H, ArH), 7.50 (d, 1H, ArH), 7.30 (d, 1H, ArH), 3.84 (d, 2H, OCH₂), 2.5 (m, overlapping with DMSO),1.50 (m, 2H, alkyls), 1.30 (m, 1H, alkyl), 0.91 (t, 3H, CH₃), 0.6 (m, 2H, alkyls), 0.5 (m, 2H, alkyls).

### EXAMPLE 4

### 6-Chloro4-(cyclopentyloxy)-3-propyl-2(1H)-quinolinone

The title compound was prepared from 6-chloro-4-hydrox-3-propyl-2(1H)-quinolinone by the same method used in Example 2 except the reaction was heated in a 100° C. oil bath for 32 h. The crude product was isolated as before. The product was purified by chromatography on silica gel (4 by 7 cm column) eluted with hexane/ethyl acetate (2:1, v/v). A 20% yield was obtained. MS (ES+): m/z 306.0 (M+1); ¹H NMR (DMSO-d₆) δ 11.8 (bs, 1H, NH), 7.56 (s, 1H, ArH), 7.49 (d, 1H, ArH), 7.28 (d, 1H, ArH), 4.70 (m, 1H, OCH), 2.5 (m, overlapping with DMSO), 1.80 (m, 6H, alkyls), 1.60 (m, 2H, CH₂s), 1.50 (m, 2H, CH₂), 0.88 (t, 3H, CH₃).

### EXAMPLE 5

### 6-Chloro-4(cyclohexylmethoxy)-3-propyl-2(1H)-quinolinone

The title compound was prepared from 6-chloro-4-hydrox-3-propyl-2(1H)-quinolinone by the same method used in example 2 except the reaction was heated in a 50° C. oil bath for 9 h followed by heating in a 75° C. oil bath for 4 h. The crude product was isolated as before. The product was purified by chromatography on silica gel (4 by 7 cm column) eluted with chloroform/methanol (97:3, v/v). A 10% yield was obtained. MS (ES+): m/z 334.0 (M+1); ¹H NMR (DMSO-d₆) δ 11.8 (bs, 1H, NH), 7.61 (s, 1H, ArH), 7.53 (d, 1H, ArH), 7.34 (d, 1H, ArH), 3.80 (m, 2H, OCH₂), 2.5 (m, overlapping with DMSO), 2.0-0.9 (m, 17H, alkyls).

### EXAMPLE 6

### 6-Chloro-4-(1,3-dioxolan-2-ylmethoxy)-3-propyl-2(1H)-quinolinone

The title compound was prepared from 6-chloro-4-hydrox-3-propyl-2(1H)-quinolinone by the method used in Example 2 except the reaction was heated in a 60° C. oil bath for 8 h followed by heating in a 100° C. oil bath for 7 h. The crude product was isolated as before. The product was purified by chromatography on silica gel (4 by 7 cm column) eluted with chloroform/methanol (97:3, v/v). A 7% yield was obtained. MS (ES+): m/z 324.0 (M+1); ¹H NMR (DMSO-d₆) δ 11.8 (bs, 1H, NH), 7.84 (s, 1H, ArH), 7.65 (d, 1H, ArM), 7.32 (d, 1H, ArH), 5.31 (m, 1H, CH), 4.0 (m, 6H, OCH₂), 2.5 (m, overlapping with DMSO), 1.5 (m, 2H, CH₂), 0.95 (t, 3H, CH₃).

### EXAMPLE 7

### 4-Benzyloxy-6-chloro-3-propyl-2(1H)-quinolinone

The title compound was prepared from 6-chloro-4-hydrox-3-propyl-2(1H)-quinolinone by the same method used in Example 2 except the reaction was heated in a 50° C. oil bath for 4 h. The crude product was isolated as before. The product was purified by chromatography on silica gel (4 by 7 cm column) eluted with chloroform/methanol (97:3, v/v). A 5% yield was obtained. MS (ES+): m/z 328.0 (M+1); ¹H NMR (DMSO-d₆) δ 11.8 (bs, 1H, NH), 7.55-7.28 (m, 8H, ArH), 5.04 (s, 2H, CH2), 2.5 (m, overlapping with DMSO), 1.44 (m, 2H, CH₂), 0.83 (t, 3H, CH₃).

### EXAMPLE 8

### 6-Chloro-4-(cyclobutyloxy)-3-propyl-2(1H)-quinolinone

The title compound was prepared from 6-chloro-4-hydrox-3-propyl-2(1H)-quinolinone by the method used in Example 2 except the reaction was heated in a 70° C. oil bath for 7 h. Sodium hydride (0.04 g) was added and the reaction heated in a 120° C. oil bath for 24 h. The crude product was isolated as before. The product was purified by recrystallization from ethyl acetate to give a 27% yield. MS (ES+): m/z 292.0 (M+1); ¹H NMR (DMSO-d₆) δ 11.8 (bs, 1H, NH), 7.56 (s, 1H, ArH), 7.53 (d, 1H, ArH), 7.32 (d, 1H, ArH), 4.46 (m, 1H, OCH), 2.5 (m, overlapping with DMSO), 2.3 (m, 3H, alkyls), 1.7 (m, 1H, alkyl), 1.50 (m, 4H, CH₂), 0.95 (t, 3H, CH₃).

### EXAMPLE 9

### 6-Chloro-4-(cyclohexyloxy)-3-isopropyl-2(1H)-quinolinone

A. 6-Chloro-4-hydroxy-3-isopropyl-2(1H) quinolinone

The title compound was prepared from 4-chloroaniline and diethyl isopropylmalonate by the method used in Example 1. MS (ES+): m/z 238.0 (M++H); 1H NMR (DMSO-d6) δ 11.3 (bs, 1H, OH), 10.0 (bs, 1H, NH), 7.87 (s, 1H, ArH), 7.44 (d, 1H, ArH), 7.22 (d, 1H, ArH), 2.5 overlapping with DMSO), 1.22 (m, 6H, CH₃).

B. 6-Chloro-4-(cyclohexyloxy)-3-isopropyl-2(1H)-quinolinone

The title compound was prepared from 6-chloro-4-hydroxy-3-isopropyl-2(1H)-quinolinone by the method outlined in Example 1. MS (ES+): m/z 320.0 (M+1); ¹H NMR DMSO-d₆) δ11.6 (bs, 1H, NH), 7.59 (s, 1H, ArH), 7.46 (d, 1H, ArH), 7.25 (d, 1H, ArH), 3.95 (m, 1H, CH), 1.95 (m, 2H, CH₂), 1.70 (m, 2H, CH₂), 1.50 (m, 3H, CH₂), 1.28-1.20 (m, 10H, alkyls).

### EXAMPLE 10

### 4-Cyclopentyloxy-6-methyl-3-propyl-2(1H)-quinolinone

A. 4-Hydroxy-6-methyl-3-propyl-2(1H)-quinolinone

The title compound was prepared from p-toluidine by the method outlined in Example 1 in 75% yield. MS (ES+): m/z 218.0 (M+1); ¹H NMR ()MSO-d₆) δ 11.3 (bs, 1H, OH), 9.8 (bs, 1H, NH), 7.62 (s, 1H, ArH), 7.21 (d, 1H, ArH), 7.09 (d, 1H, ArH), 2.5 (m, overlapping with DMSO), 2.31 (s, 3H, CH₃), 1.40 (m, 2H, CH₂), 0.86 (t, 2H, CH₃).

4-Cyclopentyloxy-6-methyl-3-propyl-2(1H)-quinolinone

The title compound was prepared from 4-hydroxy-6-methyl-3-propyl-2(1H)-quinolinone using the method employed in Example 2 except the reaction was heated for 2 h and a second portion of sodium hydride (0.025 g, 0.50 eq) was added again. The reaction was heated overnight. Sodium hydride (0.025 g, 0.50 eq) was added again and heating at 60° C. was continued another four hours. The product was isolated as before. Final purification was accomplished by chromatography on silica gel eluted with chloroform/methanol (98:2, v/v). A 2% yield was obtained. MS (ES+): m/z 286.0 (M+1); ¹H NMR (DMSO-d₆) δ 11.5 (bs, 1H, NH), 7.41 (s, 1H, ArH), 7.24 (d, 1H, ArH), 7.15 (d, 1H, ArH), 4.70 (m, 1H, OCH), 2.5 (m, overlapping with DMSO), 2.32 (s, 3H, CH₃), 1.9-1.2 (m, 12H, CH₂s), 0.88 (t, 3H, CH₃).

### EXAMPLE 11

### 4-Cyclopentyloxy-6-methoxy-3-propyl-2(1H)-quinolinone

A. 4-Hydroxy-6-methoxy-3-propyl-2(1H)-quinolinone

The title compound was prepared from 4-methoxyaniline by the method outlined in Example 1 in 72% yield. MS (ES+): m/z 234.0 (M+1); ¹ H NMR (DMSO-d₆) δ11.2 (bs, 1H, OH), 9.9 (bs, 1H, NH), 7.38(s, 1H, ArH), 7.19 (d, 1H, ArH), 7.1 (d, 1H, ArH), 3,80 (s, 3H, OCH₃), 2.5 (m, overlapping with DMSO), 1.40 (m, 2H, CH₂), 0.92 (t, 2H, CH₃).

B. 4-Cyclopentyloxy-6-methoxy-3-propyl-2(1H)-quinolinone

The title compound was prepared from 4-hydroxy-6-methoxy-3-propyl-2(1H)-quinolinone using the method employed in Example 10 except the reaction was heated in a 100° C. oil bath for a total of 24 h. A 16% yield was obtained. MS (ES+): m/z 302.0 (M+1); ¹H NMR (DMSO-d₆) δ 11.5 (bs, 1H, NH), 7.20 (s, 1H, ArH), 7.10 (d, 1H, ArH), 7.06 (d, 1H, ArH), 4.70 (m, 1H, OCH), 3.75 (s, 3H, OCH₃), 2.5 (m, overlapping with DMSO), 1.80 (m, 6H, CH₂s), 1.60 (m, 2H, CH₂s), 1.50 (m, 2H, CH₂), 0.88 (t, 3H, CH₃).

### EXAMPLE 12

### 4-(Cyclopentyloxy)-3-propyl-6-trifluoromethoxy-2(1H)-quinolinone

A. 4-Hydroxy-3-propyl-6-trifluoromethoxy-2(1H)-quinolinone

The title compound was prepared from 4-(trifluoromethoxy)aniline by the method outlined in Example 1 in 40% yield. MS (ES+): m/z 288.0 (M+1); ¹H NMR (DMSO-d₆) δ 11.5 (bs, 1H, OH), 10.3 (bs, 1H, NH), 7.8 (s, 1H, ArH), 7.5 (d, 1H, ArH), 7.3 (d, 1H, ArH), 2.5 (m, overlapping with DMSO), 1.40 (m, 2H, CH₂), 0.92 (t, 2H, CH₃).

B. 4-(Cyclopentyloxy)-3-propyl-6-trifluoromethoxy-2(1H)-quinolinone

The title compound was prepared from 4-hydroxy-3-propyl-6-trifluoromethoxy-2(1H)-quinolinone using the method employed in Example 10. A 9% yield was obtained. MS (ES+): m/z 356.0 (M+1); ¹H NMR (DMSO-d₆) δ 11.8 (bs, 1H, NH), 7.50 (s, 1H, ArH), 7.46 (d, 1H, ArH), 7.34 (d, 1H, ArH), 4.68 (m, 1H, OCH), 2.5 (m, overlapping with DMSO), 1.80 (m, 6H, CH₂s), 1.60 (m, 2H, CH₂s), 1.50 (m, 2H, CH₂), 0.88 (t, 3H, CH₃).

### EXAMPLE 13

### 4-Cyclopentyloxy-2-oxo-3-propyl-1,2-dihydro-6-guinolinecarbonitrile

A. 4-Hydroxy-2-oxo-3-propyl-1,2-dihydro-6-guinolinecarbonitrile

The title compound was prepared from 4-aminobenzonitrile by the method outlined in Example 1 in 77% yield. MS (ES+): m/z 229.0+1); ¹H NMR (DMSO-d₆) δ 11.7 (bs, 1H, OH), 10.5 (bs, 1H, NH), 8.23 (s, 1H, ArH), 7.77 (d, 1H, ArH), 7.33 (d, 1H, ArH), 2.5 (m, overlapping with DMSO), 1.4 (m, 2H, CH₂), 0.87 (t, 2H, CH₃).

B. 4-Cyclopentyloxy-2-oxo-3-propyl-1,2-dihydro-6-guinolinecarbonitrile

The title compound was prepared from 4-hydroxy-2-oxo-3-propyl-1,2-dihydro-6-quinolinecarbonitrile using the method employed in Example 10. A 15% yield was obtained. MS (APCH−): m/z 295.0 (M−1); ¹H NMR (DMSO-d₆) δ 12.0 (bs, 1H, NH), 8.00 (s, 1H, ArH), 7.82 (d, 1H, ArH), 7.37 (d, 1H, ArEH), 4.74 (m, 1H, OCH), 2.5 (m, overlapping with DMSO), 1.80 (m, 6H, CH₂s), 1.60 (m, 2H, CH₂s), 1.50 (m, 2H, CH₂), 0.88 (t, 3H, CH₃).

### EXAMPLE 14

### 6-Bromo-4-(cyclopentyloxy)-3-propyl-2(1H)-quinolinone

A. 6-Bromo-4-hydroxy-3-propyl-2(1H)-quinolinone

The title compound was prepared from 4-bromoaniline by the method outlined in Example 1 in 58% yield. MS (ES+): m/z 284.0 (M+1); ¹H NMR (DMSO-d₆) δ 11.4 (bs, 1H, OH), 10.2 (bs, 1H, NH), 7.95 (s, 1H, ArH), 7.54 (d, 1H, ArH), 7.15 (d, 1H, ArH), 2.5 (m, overlapping with DMSO), 1.40 (m, 2H, CH₂), 0.86 (t, 2H, CH₃).

B. 6-Bromo-4-(cyclopentyloxy)-3-propyl-2(1H)-quinolinone

The title compound was prepared from 6-bromo-4-hydroxy-3-propyl-2(1H)-quinolinone using the method employed in Example 10. A 7% yield was obtained. MS (ES+): m/z 352.0 (M+1); ¹H NMR (DMSO-d₆) δ 11.8 (bs, 1H, NH), 7.70 (s, 1H, ArH), 7.60 (d, 1H, ArH), 7.21 (d, 1H, ArH, 4.69 (m, 1H, OCH), 2.5 (m, overlapping with DMSO), 1.80 (m, 6H, alkyls), 1.60 (m, 2H, CH₂), 1.50 (m, 2H, CH₂), 0.88 (t, 3H, CH₃).

### EXAMPLE 15

### 4-Cyclopentyloxy-3-propyl-2(1H)-quinolinone

A. 4-Hydroxy-3-propyl-2(1H)-quinolinone

The title compound was prepared from aniline by the method outlined in Example 1 in 73% yield. MS (ES+): m/z 204.0 (M+1); ¹H NMR (DMSO-d₆) δ 11.2 (bs, 1H, OH), 9.94 (bs, 1H, NH), 7.82 (d, 1H, ArH), 7.39 (t, 1H, ArH), 7.19 (d, 1H, ArH), 7.09 (t, 1H, ArH), 2.5 (m, overlapping with DMSO), 1.40 (m, 2H, CH₂), 0.86 (t, 2H, CH₃).

B. Cyclopentyloxy-3-propyl-2(1H)-quinolinone

The title compound was prepared from 4-hydroxy-3-propyl-2(1H)-quinolinone using the method employed in Example 10. A 9% yield was obtained. MS (ES+): m/z 272.0 (M+1); ¹H NMR (DMSO-d₆) δ 11.6 (bs, 1H, NH), 7.64 (d, 1H, ArH), 7.42 (t, 1H, ArH), 7.25 (d, 1H, ArH), 7.15 (t, 1H, ArH), 4.70 (s, 1H, OCH), 2.5 (m, overlapping with DMSO), 1.80 (m, 6H, alkyls), 1.60 (m, 2H, CH₂), 1.50 (m, 2H, CH₂), 0.88 (t, 3H, CH₃).

### EXAMPLE 16

### 6-Chloro-4-cyclobutylmethoxy-3-isopropyl-2(1H)-quinolinone

The title compound was prepared from 6-Chloro-4-hydroxy-3-isopropyl-2(1H)-quinolinone using the method employed in Example 10. A 22% yield was obtained. MS (EI+): m/z 305.0 (M+); ¹H NMR (CDCl₃) δ 12.5 (bs, 1H, NH), 7.70 (s, 1H, ArH), 7.40 (d, 1H, ArH), 7.30 (d, 1H, ArH), 3.94 (d, 2H, OCH₂), 3.45 (m, 1H, CH), 2.91 (m, 1H, CH), 2.22 (m, 2H, alkyls), 1.99 (m, 4H, alkyls), 1.45 (d, 6H, CH₃).

### EXAMPLE 17

### 6-Chloro-4-(cyclopentyloxy)-3-ethyl-2(1H)-quinolinone

A. 6-Chloro-3-ethyl-4-hydroxyl-2(1H)-quinolinone

The title compound was prepared from 4-chloroaniline and diethyl ethylnalonate by the method outlined in Example 1 in 69% yield. MS (ES+): m/z 224.0 (M+1);¹H NMR (DMSO-d₆) δ 11.4 (bs, 1H, OH), 10.2 (bs, 1H, NH), 7.82 (s, 1H, ArH), 7.44 (d, 1H, ArH), 7.21 (d, 1H, ArH), 2.5 (m, overlapping with DMSO), 0.97 (t, 2H, CH₃).

B. 6-Chloro-4-(cyclopentyloxy)-3-ethyl-2(1H)-quinolinone

The title compound was prepared from 6-chloro-3-ethyl-4-hydroxyl-2(1H)-quinolinone using the method employed in Example 10. A 12% yield was obtained. MS (ES+): m/z 292.0 (M+1); ¹H NMR (MSO-d₆) δ 11.8 (bs, 1H, NH), 7.62 (s, 1H, ArH), 7.5 (d, 1H, ArH), 7.33 (d, 1H, ArH), 4.8 (m, 1H, OCH), 2.5 (m, overlapping with DMSO), 1.9 (m, 6H, alkyls), 1.60 (m, 2H, CH₂s), 1.12 (t, 3H, CH₃).

### EXAMPLE 18

### 3-(sec-Butyl)-6-chloro-4-(cyclopentyloxy)-2(1H)-quinolinone

A. 3-(sec-Butyl)-6-chloro-4-hydroxyl-2(1H)-quinolinone

The title compound was prepared from 4-chloroaniline and diethyl sec-butylmalonate by the method outlined in Example 1 in 35% yield. MS (ES+): m/z 252.0 (M+H); ¹H NMR (DMSO-d₆) δ 11.3 (bs, 1H, OH), 10.0 (bs, 1H, NH), 7.87 (s, 1H, ArH), 7.44 (d, 1H, ArH), 7.2 (d, 1H, ArH), 3.12 (m, 1H, CH), 1.87 (m, 1H, CH), 1.6 (m, 1H, CH), 1.2 (d, 3H, CH₃), 0.7 (m, 3H, CH₃).

B. 3-(sec-Butyl)-6-chloro-4(cyclopentyloxy)-2(1H)-quinolinone

The title compound was prepared from 3-(sec-butyl)-6-chloro-4-hydroxyl-2(1H)-quinolinone using the method employed in Example 10. A 2% yield was obtained. MS (ES+): m/z 320.0 (M+1); ¹H NMR (DMSO-d₆) δ 11.6 (bs, 1H, NH), 7.56 (s, 1H, ArH), 7.49 (d, 1H, ArH), 7.28 (d, 1H, ArH), 4.60 (m, 1H, OCH), 3.0 (m, 1H, CH), 1.80 (m, 8H, CH₂), 1.60 (m, 2H, Ch₂), 1.25 (d, 3H, CH₃), 0.73 (t, 3H, CH₃).

### EXAMPLE 19

### 6-Chloro-4-(cyclopentyloxy)-3-isobutyl-2(1H)-quinolinone

A. 6-Chloro-4-hydroxyl-3-isobutyl-2(1H)-quinolinone

The title compound was prepared from 4-chloroaniline and diethyl isobutylmalonate by the method outlined in Example 1. Reaction time was 3 d. A 51% yield was obtained. MS (ES+): m/z 252.0 (M+H); ¹H NMR (DMSO-d₆) δ 11.4 (bs, 1H, OH), 10.1 (bs, 1H, NH), 7.83 (s, 1H, ArH), 7.44 (d, 1H, ArH), 7.22 (d, 1H, ArH), 2.5 (m, overlapping with DMSO), 1.86 (m, 1H, CH), 0.82 (m, 6H, CH₃).

B. 6-Chloro-4-(cyclopentyloxy)-3-isobutyl-2(1H)-quinolinone

The title compound was prepared from 6-chloro-4-hydroxyl-3-isobutyl-2(1H)-quinolinone using the method employed in Example 10. A 13% yield was obtained. MS (ES+): m/z 320.0 (M+1); ¹H NMR (CDCl₃) δ 10.4 (bs, 1H, NH), 7.70 (s, 1H, ArH), 7.35 (d, 1H, ArH), 7.15 (d, 1H, ArH), 4.73 (m, 1H, OCH), 2.60 (d, 2H, CH₂), 2.08-1.54 (m, 9H, alkyls), 0.90 (t, 6H, CH₃).

### EXAMPLE 20

### 6-Chloro-4-(cyclohexyloxy)-3-propyl-2(1H)-quinolinethione

6-Chloro-4-(cyclohexyloxy)-3-propyl-2(1H)-quinolinone (0.025 g, 0.078 mmol) and Lawesson's reagent (0.038 g, 0.094 mmol) were combined in toluene (5 mL) and heated in a 100° C. oil bath for 2 h. The solvent was removed in vacuo and the product was isolated by chromatography on silica gel (4 by 7 cm column) eluted with hexane/ethyl acetate (9:1, v/v). A 19% yield was obtained. MS APCI−): m/z 334.0 (M−1); ¹H NMR (DMSO-d₆) δ 13.6 (bs, 1H, NH), 7.78 (s, 1H, ArH), 7.67 (s, 2H, ArH), 3.95 (m, 1H, CH), 4.17, (m, 1H, CH), 2.92 (m, 2H, CH₂), 2.0-0.9 (m, 15H, alkyls).

### EXAMPLE 21

### 6-Chloro-4-(cyclobutylmethoxy)-3-propyl-2(1H)-quinolinethione

The title compound was prepared from 6-Chloro-4-(cyclobutylmethoxy)-3-propyl-2(1H)-quinolinone by the method outlined in Example 20. The product was isolated by chromatography on silica gel (4 by 7 cm column) eluted with chloroform/methanol (97:3, v/v, 2 times). An 8% yield was obtained. MS (ES+): m/z 322.0 (M+1); ¹H NMR (DMSO-d₆) δ 13.6 (bs, 1H, NH), 7.67 (s, 1H, ArH), 7.62 (s, 2H, ArH), 4.00 (d, 2H, OCH₂), 2.83 (t, 3H, alkyls), 2.1 (m, 2H, alkyls), 1.9 (m, 4H, alkyl), 1.55 (m, 2H, alkyls), 0.90 (t, 3H, CH₃).

### EXAMPLE 22

### 3-(sec-Butyl)-6-chloro-4-(cyclopentyloxy)-2(1H)-quinolinethione

The title compound was prepared from 3-(sec-butyl)-6-chloro-4-(cyclopentyloxy)-2(1H)-quinolinone by the method outlined in Example 20. The reaction was heated for 23 h. The product was isolated by chromatography on silica gel (4 by 7 cm column) eluted with chloroform/methanol (97:3, v/v) followed by chromatography on silica gel (4 by 7 cm column) eluted with hexane/ethyl acetate (9:1, v/v). A 4% yield was obtained. MS (ES+): m/z 336.0 (M+1); ¹H NMR (DMSO-d₆) δ 13.4 (bs, 1H, NH), 7.68 (s, 1H, Ard), 7.61 (d, 2H, ArH), 4.72 (m, 1H, OCH), 2.2-0.7 (m, alkyls).

### EXAMPLE 23

### 6-Chloro-4-(cyclohexylsulfanyl)-3-propyl-2(1H)-quinolinone

A. 2,4-Dibromo-6-chloro-3-propylquinoline

6-Chloro-4-hydroxy-3-propyl-2(1H)-quinolinone (1.0 g, 4.2 mmol) and phosphorus tribromide (12 mL) were combined and heated to reflux in a 150° C. oil bath overnight. The reaction was poured onto ice water (500 mL) and the product precipitated. The solid was collected by filtration and the product was recrystallized from ethanol giving a 40% yield. MS (ES+): m/z 364.0 (M+1); ¹H NMR DMSO-d₆) δ 8.09 (s, 1H, ArH), 8.00 (d, 1H, ArH), 7.83 (d, 1H, ArH), 3.05 (m, 2H, CH₂), 1.60 (m, 2H, CH₂), 1.00 (t, 3H, CH₃).

B. 6-Chloro-4-bromo-3-propyl-2(1H)-quinolinone 2,4-Dibromo-6-chloro-3-propylquinoline (0.55 g, 1.5 mmol) was heated to reflux in a solution of acetic acid and water (80 mL, 2:1, v/v) for 22 h. The reaction was allowed to cool to rt. The product was collected by filtration giving a 67% yield. MS (ES+): m/z 302.0 (M+1); ¹H NMR (DMSO-d₆) δ 12.2 (bs, 1H, NH), 7.76 (s, 1H, ArH), 7.55 (d, 1H, ArH), 7.31 (d, 1H, ArH), 2.71 (t, 2H,CH₂), 1.51 (m, 2H, CH₂), 0.93 (t, 3H, CH₃).

C. 6-Chloro-4-(cyclohexylsulfanyl)-3-propyl-2(1H)-quinolinone

Cyclohexyl mercaptan (0.98 mL, 8.0 mmol) was dissolved in DMF (5 mL) and the solution cooled to 0° C. Sodium hydride (0.16 g, 4.0 mmol) was added and the reaction stirred for 45 min. 6-Chloro-4-bromo-3-propyl-2(1H)-quinolinone (0.3 g, 1 mmole) was dissolved in DMF (5 mL) and added to the above solution. The reaction was heated to 140° C. for 20 h under a N₂ atmosphere. The reaction was cooled to rt. A precipitate was filtered and the solvent removed from the filtrate in vacuo. The residue was dissolved in ethyl acetate and a few drops of water added. The product precipitated and was collected by filtration. A second crop was obtained from the filtrate by recrystallization from ethyl acetate to give a total yield of 62%. MS (ES+): m/z 336.0 (M+1); ¹H NMR (CDCl₃) δ 10.9 (bs, 1H, NM), 8.21 (s, 1H, ArH), 7.41 (d, 1H, ArH), 7.20 (d, 1H, ArH), 3.05 (m, 2H, CH₂), 3.0 (m, 1H, SCH), 1.9 (m, 2H, CH₂), 1.78 (m, 2H, CH₂), 1.57 (m, 2H, CH₂), 1.25(m, 6H, CH₂), 1.05 (t, 3H, CH₃).

### EXAMPLE 24

### 6-Chloro-4-(cyclohexylsulfinyl)-3-propyl-2(1H)-quinolinone

6-Chloro-4-(cyclohexylsulfanyl)-3-propyl-2(1H)-quinolinone (0.08 g, 0.24 mmol) was dissolved in iPrOH (10 mL). 3-Chloroperbenzoic acid (0.16 g, 0.53 mmol) dissolved in iPrOH (5 mL) was added and the reaction stirred rt for 1 h. The reaction mixture was washed with saturated sodium bicarbonate solution and dried with sodium sulfate. The volume of iPrOH was reduced in vacuo and the product crystallized giving a 60% yield. MS (ES+): m/z 352.0 (M+1); ¹H NMR (DMSO-d₆) δ 12.3(bs, 1H, NH), 8.8 (bs, 1H, ArH), 7.57(d, 1H, ArH), 7.37 (d, 1H, ArH), 2.8-0.94 (m, alkyls).

### EXAMPLE 25

### 4Cyclopentyloxy-6-fluoro-3-propyl-2(1H)-quinolinone

A. 6-Fluoro-4-hydroxyl-3-propyl-2(1H)-quinolinone

The title compound was prepared from 4-fluoroaniline by the method in Example 1. A 56% yield was obtained. MS (ES+): m/z 222.0 (M+1); ¹H NMR (DMSO-d₆) δ 11.3 (bs, 1H, OH), 10.0 (bs, 1H, NH), 7.87 (d, 1H, ArH), 7.42 (dd, 1H, ArH), 7.22 (d, 1H, ArH), 2.5 (m, overlapping with DMSO), 1.40 (m, 2H, CH₂), 0.86 (t, 3H, CH₃).

B. 4-Cyclopentyloxy-6-fluoro-3-propyl-2(1H)-quinolinone

The title compound was prepared from 6-fluoro-4-hydroxyl-3-propyl-2(1H)-quinolinone by the method used in Example 8 except the oil bath temperature was 50° C. A 22% yield was obtained. MS (ES+): m/z 290.0 (M+1); ¹H NMR (DMSO-d₆) δ 11.7 (bs, 1H, NH), 7.32 (m, 3H, ArH), 4.70 (m, 1H, OCH), 2.5 (m, overlapping with DMSO), 1.80 (m, 6H, alkyls), 1.60 (m, 2H, CH₂s), 1.50 (m, 2H, CH₂), 0.88 (t, 3H, CH₃).

### EXAMPLE 26

### 4-Cyclobutylmethoxy-6-fluoro-3-propyl-2(1H)-quinolinone

The title compound was prepared from 6-fluoro-4-hydroxyl-3-propyl-2(1H)-quinolinone by the method used in Example 8 except the oil bath temperature was 60° C. A 40% yield was obtained. MS (ES+): m/z 290.0 (M+H); ¹H NMR CDMSO-d₆) δ 11.8 (bs, 1H, NH), 7.32 (m, 3H, ArH), 4.00 (d, 2H, OCH₂), 2.86 (m, 1H, CH), 2.14 (m, 2H, alkyls), 1.95 (m, 4H, alkyls), 1.65 (m, 2H, CH₂), 0.95 (t, 3H, CH₃).

### EXAMPLE 27

### 3-sec-Butyl-4-(cyclobutylmethoxy)-6-fluoro-2(1H)-quinolinone

A. 3-sec-Butyl-6-fluoro-4-hydroxyl-2(1H)-quinolinone

The title compound was prepared from 4-fluoroaniline (10 g, 80 mmol) and diethyl sec-butylmalonate by the method used in Example 1. Reaction time was 5 d. MS (ES+): m/z 236.0 (M+H); ¹H NMR (DMSO-d₆) δ 11.2 (bs, 1H, OH), 9.95 (bs, 1H, NH), 7.60 (d, 1H, ArH), 7.31-7.20 (m, 2H, ArH), 3.13 (m, 1H, CH), 1.9 (m, 1H, CH), 1.6 (m, 1H, CH), 1.2 (d, 3H, CH₃), 0.7 (m, 3H, CH₃).

B. 3-sec-Butyl-4-(cyclobutylmethoxy)-6-fluoro-2(1H)-quinolinone

The title compound was prepared from 3-sec-butyl-6-fluoro-4-hydroxyl-2(1H)-quinolinone by the method used in Example 26. A 35% yield was obtained. MS (ES+): m/z 304.0 (M+1); ¹H NMR (DMSO-d₆) δ 11.6 (bs, 1H, NH), 7.37 (m, 3H, ArH), 3.92 (d, 2H, OCH₂), 3,08 (m, 1H), 2.87 (m, 1H), 2.1 (m, 2H, alkyls), 1.9 (m, 4H, alkyl), 1.31 (d, 3H, CH₃), 1.7 (m, 1H), 0.80 (t, 3H, CH₃).

### EXAMPLE 28

### 4-(Cyclobutyhnethoxy)-6-fluoro-3-isopropyl-2(1H)-quinolinone

A. 6-Fluoro-4-hydroxyl-3-isopropyl-2(1H)-quinolinone

The title compound was prepared from 4-fluoroaniline (10 g, 80 mmol) and diethyl isopropylmalonate by the method in Example 1. Reaction time was 5 d. A 40% yield was obtained. MS (ES+): m/z 222.0 (M+H); ¹H NMR (DMSO-d₆) δ 10.9 (bs, 1H, OH), 10.1 (bs, 1H, NH), 7.7 (d, 1H, ArH), 7.4-7.20 (m, 2H, ArH), 3.3 (m, overlapping with DOH), 1.29 (d, 6H, CH₃).

B. 4-(Cyclobutylmethoxy)-6-fluoro-3-isopropyl-2(1H)-quinolinone

The title compound was prepared from 6-fluoro-4-hydroxyl-3-isopropyl-2(1H)-quinolinone by the method used in Example 26. A 2% yield was obtained. MS (EI+): m/z 305.0 (M+); ¹H NMR (DMSO-d₆) δ 11.7 (bs, 1H, NH), 7.4-7.3 (m, 3H, ArH), 3.94 (d, 2H, OCH₂), 2.9 (m 1H, CH), 2.1 (m, 2H, alkyls), 1.93 (m, 4H, alkyls), 1.93 (d, 6H, CH₃).

### EXAMPLE 29

### 4-Cyclopentyloxy-6-fluoro-3-propyl-2(1H)-quinolinethione

The title compound was prepared from 4-cyclopentyloxy-6-fluoro-3-propyl-2(1H)-quinolinone by the method in Example 20. A 30% yield was obtained. MS (ES+): m/z 306.0 (M+1); ¹H NMR (DMSO-d₆) δ 13.6 (bs, 1H, NH), 7.66 (m, 1H, ArH), 7.5-7.4 (m, 2H, 4.78 (m, 1H, OCH), 2.86 (m, 2H), 1.80 (m, 6H, alkyls), 1.60 (m, 4H, CH₂s), 0.88 (t, 3H, CH₃).

### EXAMPLE 30

### 4-(Cyclobutylmethoxy)-6-fluoro-3-propyl-2(1H)-quinolinethione

The title compound was prepared from 4-(cyclobutylmethoxy)-6-fluoro-3-propyl-2(1H)-quinolinone by the method used in Example 20. A 33% yield was obtained. MS (ES+): m/z 306.0 (M+1); ¹H NMR (DMSO-d₆) δ 13.6 (bs, 1H, NH), 7.66 (m, 1H, ArH), 7.53-7.42 (m, 2H, ArH), 4.0 (d, 2H, OCH₂), 2.84 (m, 2H), 2.2-1.8 (m, 6H, alkyls), 1.60 (m, 3H, CH₂), 0.88 (t, 3H, CH₃).

### EXAMPLE 31

### 4(Cyclobutylnethoxy)-6-fluoro-3-isopropyl-2(1H)-quinolinethione

The title compound was prepared from 4-(cyclobutylmethoxy)-6-fluoro-3-isopropyl-2-(1H)-quinolinone by the method used in Example 20. A 6% yield was obtained. MS (ES+): m/z 306.0 (M+1); ¹H NMR (DMSO-d₆) δ 13.6 (bs, 1H, NH), 7.66 (m, 1H, ArH), 7.50 (m, 1H, ArH), 7.37 (d, 1H, ArH), 4.05 (m, 1H, CH), 3.97 (d, 2H, OCH₂), 2.91 (m, 1H, CH), 2.1 (m, 2H, alkyls), 1.9 (m, 4H, CH₂), 1.3 (t, 6H, CH₃).

### EXAMPLE 32

### 6-Chloro-4-(isobutylamino)-3-propyl-2(1H)-quinolinone

2,4-Dibromo-6-chloro-3-propylquinoline (0.037 g, 0.12 mmol) was dissolved in ethanol (2 mL) and isobutyl amine (1 mL) and heated in a 150° C. oil bath for 14 d. The solvents were removed in vacuo. The product was isolated by chromatography on silica gel (4 by 7 cm column) eluted with ethyl acetate/hexane (1:2, v/v). MS (ES+): m/z 293.0 (M+1); ¹H NMR (CDCl₃) δ 11.8 (bs, 1H, NH), 7.71 (s, 1H, ArH), 7.35 (d, 1H, ArH), 7.18 (d, 1H, ArH), 3.86 (m, 1H, NH), 3.12 (t, 2H, CH₂), 2.65 (m, 2H, CH₂), 1.9 (m, 1H, CH), 1.6 (m, 2H, CH₂), 0.95 (t, 9H, CH₃).

### EXAMPLE 33

### 6-Chloro-4-(cyclobutylmethoxy)-3-ethoxy-2(1.H)-quinolinone

A. Ethoxyacetyl chloride

Ethoxy acetic acid (10 g, 96 mmol) was added to thionyl chloride (11 g, 92 mmol) and heated in a 70° C. oil bath for 1 h. The product, purified by distillation at 135° C. from a 190° C. oil bath, was suitable for further use.

B. Methyl 5-chloro-2-[(2-ethoxyacetyl)amino]benzoate

Methyl 2-amino-5-chlorobenzoate (1.0 g, 5.4 mmol) was dissolved in dichloromethanc (10 mL). Ethoxyacetyl chloride (1.0 g, 8.1 mmol) was added followed by TEA (2.2 mL, 16 mmol). The reaction was stirred at rt 5 min. Water was added and the mixture transferred to a separatory funnel. The organic layer was washed with saturated ammonium chloride (2×). The solvent was removed in vacuo and the product was isolated by chromatography on silica gel (4×15 cm column) eluted with chloroform/ methanol (98:2, v/v-500 mL followed by 95:5, 300 mL). A 55% yield was obtained. MS (ES+): m/z 272.0 (M+1); ¹H NMR (CDCl₃) δ 11.1 (bs, 1H, NH), 8.78 (d, 1H, ArH), 8.01 (s, 1H, ArH), 7.49 (d, 1H, ArH), 4.1 (s, 2H, CH₂), 3.95 (s, 3H, OCH₃), 3.65 (m, 2H, CH₂), 1.38 (m, 3H, CH₃).

C. 6-Chloro-3-ethoxy-4-hydroxy-2(1H)-quinolinone

Methyl 5-chloro-2-[(2-ethoxyacetyl)amino]benzoate (0.20 g, 0.74 mmol) was boiled in toluene to remove water. The excess toluene was removed in vacuo. The residue was in THF (8 mL) and chilled to −78° C. Bis(trimethylsilyl) potassium amide (4.4 mL of a 0.5 M solution in toluene, 2.2 mmol) was added over 5 min. The reaction was stirred for 5 min and transferred to a 0° C. bath. The reaction was stirred for 30 min then allowed to warm to rt and stir overnight. The reaction was poured onto ice water (15 mL) and extracted with ethyl acetate (20 mL). The solvent was removed in vacuo and the product was isolated by filtration through silica gel (10 g) eluted with ethyl acetate/hexane (1:4, v/v). A 22% yield was obtained. MS (ES+): m/z 240.0 (M+1); ¹H NMR (CDCl₃) δ 11.6 (bs, 1H, NH), 10.6 (bs, 1H, OH), 7.68 (s, 1H, ArH), 7.42 (d, 1H, ArH), 7.22 (d, 1H, Aril), 4.04 (q, 2H, OCH₂), 1.22 (t, 2H, CH₃).

D. 6- Chloro-4-(cyclobutylmethoxy)-3-ethoxy-2(1H)-quinolinone

The title compound was prepared from 6-chloro-3-ethoxy-4-hydroxy-2(1H)-quinolinone by the method in Example 8 except a 60° C. oil bath was used. The reaction time was 48 h. The product was isolated by chromatography on silica gel (4×7 cm column) eluted with ethyl acetate/hexane (1:1). An 11% yield was obtained. MS (ES+): m/z 308.0 (M+1); ¹H NM (CDCl₃) δ 10.4 (bs, 1H, NH), 7.8(s, 1H, ArH), 7.36 (d, 1H, ArH), 7.14 (d, 1H, ArH), 4.42 (d, 2H, OCH₂), 4.2 (q, 2H, OCH₂), 2.81 (m, 1H, CH), 2.2-1.2 (m, alkyls), 0.87 (m, 2H).

### EXAMPLE 34

### 4-(Cyclobutylmethoxy)-3-ethoxy-6-fluoro-2(1H)-quinolinone

A. Ethyl 2-amino-5-fluorobenzoate

2-Amino-5-fluorobenzoic acid (1.0 g, 6.4 mmol) was dissolved in ethanol (15 mL) and toluene (10 mL). Several drops of concentrated H₂SO₄ were added. The reaction was refluxed with a Dean Stark trap overnight. The reaction was cooled to rt and the solvents removed in vacuo. The product was isolated by chromatography on silica gel (4×15 cm column) eluted with chloroform/ methanol (98:2, v/v-500 mL followed by 95:5, 500 mL) in 50% yield. MS (ES+): m/z 184.0 (M+1); ¹H NMR (CDCl₃) δ 7.55 (d, 1H, ArH), 7.03 (m, 1H, ArH), 6.61 (dd, 1H, ArH), 5.6 (bs, 2H, NH₂), 4.33 (q, 2H,CH₂), 1.40 (t, 3H, CH₃).

B. 4-(Cyclobutylmethoxy)-3-ethoxy-6-fluoro-2(1H)-quinolinone

The title compound was prepared from ethyl 2-amino-5-fluorobenzoate by steps I-IV in Example 33. MS (ES+): m/z 292.0 (M+1); ¹H NMR (CDCl₃) δ 11.1 (bs, 1H, NH), 7.52(d, 1H, ArH), 7.24 (m, 1H, ArH), 7.15 (m, 1H, ArH), 4.42 (d, 2H, OCH₂), 4.2 (q, 2H, OCH₂), 2.81 (m, 1H, CH), 2.2-1.2 (m, alkyls), 0.87 (m, 2H).

### EXAMPLE 35

### 6-Chloro-4-[(2-cyclopropylethynyl)oxy]-3-propyl-2(1H)-quinolinone

A. 6-Chloro-3-propyl-4-(2,2,2-trifluoroethoxy)-2(1H)-quinolinone

6-Chloro-4-hydroxy-3-propyl-2(1H)-quinolinone (1.5 g, 6.3 mmol) was dissolved in DMF (12 mL) and treated with sodium hydride (0.55 g of a 60% oil dispersion, 13 mmol, 2.1 eq.) at rt for 5 min. 2-Bromo-1,1-trifluoroethane (1.8 mL, 19.8 mmol) was added and the reaction heated in a 150° C. oil bath for 48 h. The solution was cooled to rt and poured onto ice water (70 mL). The resulting precipitate was collected by filtration. The product was isolated by filtration through silica gel (50 g) eluted with ethyl acetate/hexane (3:7, v/v). The solvents were removed in vacuo and the residue slurried in ethyl ether to give the product in 25% yield. MS (ES−): m/z 318.0 (M−1); ¹H NMR (CDCl₃) δ 11.4 (bs, 1H, NH), 7.70 (s, 1H, ArH), 7.45 (d, 1H, ArH), 7.26 (d, 1H, ArH), 4.35 (q, 2H, CH₂), 2.66 (m, 2H, CH₂), 1.66 (m, 2H, CH₂), 1.01 (t, 3H, CH₃).

B. 6-Chloro-2-[(4-methoxybenzyl)oxy]-3-propylquinolinyl 2,2,2-trifluoroethyl ether

6-Chloro-3-propyl-4-(2,2,2-trifluoroethoxy)-2(1H)-quinolinone (0.26 g, 0.80 mmol) was dissolved in DMF (3 mL). Silver (I) oxide (0.20 g, 0.86 mmol) was added followed by 4-methoxybenzyl chloride (0.135 mL,1.0 mmol) The reaction was heated in a 100° C. oil bath overnight. The oil bath temperature was raised to 145° C. for 1 h. Silver(I) oxide (0.1 g, 0.4 mmol) and 4-methoxybenzyl chloride (0.050 mL, 0.37 mmol) were added, the oil bath temperature was adjusted to 110° C., and the reaction heated for 4 h. Silver(I) oxide (0.1 g, 0.4 mmol) and 4-methoxybenzyl chloride (0.050 mL, 0.37 mmol) were again added and the reaction was heated in the 145° C. oil bath for an additional 1.5 h. The reaction was cooled to rt and filtered. The filtrate was poured into water (25 mL) and the resulting precipitate was collected by filtration. The product was isolated by filtration through silica gel (40 g) eluted with neat chloroform. The product was collected as a white solid in 47% yield. GCMS (EI): m/z 439 (M⁺), one peak.

C. 6-Chloro-4-[(2-cyclopropylethynyl)oxy]-2-[(4-methoxybenzyl)oxy]-3-propylquinoline

6-Chloro-2-[(4-methoxybenzyl)oxy]-3-propyl-4-quinolinyl 2,2,2-trifluoroethyl ether (0.14 g, 0.33 mmol) was dissolved in ether (5 mL) and chilled to −78° C. A solution cyclopropyl lithium was prepared by the reaction of cyclopropyl bromide (0.93 mL, 12 mmol) with lithium wire (0.16 g, 23 mmol) in ether (10 mL) in a 0° C. bath for 1.5 h. Cyclopropyl lithium solution (4.0 mL, 2.3 mmol) was added at −78° C. and the reaction was allowed to warm to rt overnight. The reaction was washed with saturated ammonium chloride. The organic phase was dried with magnesium sullfate, filtered and the solvent removed in vacuo. The product was purified by chromatography on silica gel (4×7 cm column) eluted with chloroform/c-hexane (1:4, v/v). A 32% yield was obtained. GCMS (CI+): m/z 439.0 (M+NH4); ¹H NMR (CDCl₃) δ 8.06 (s, 1H, ArH), 7.78 (d, 1H, ArH), 7.55 (d, 1H, ArH), 7.43 (d, 2H, ArH), 6.91 (d, 2H, ArH), 5.47 (s, 2H, CH2), 3.82 (s, 3H, OCH₃), 2.84 (m, 2H, CH₂), 1.60 (m, 2H, CH₂), 1.15 (m, 1H, CH), 0.97 (t, 3H, CH₃), 0.65 (m, 2H, CH₂), 0.55 (m, 2H, CH₂).

D. 6-Chloro-4-[(2-cyclopropylethynyl)oxy]-3-propyl-2(1H)-quinolinone

6-Chloro-4-[(2-cyclopropylethynyl)oxy]-2-[(4-methoxybenzyl)oxy]-3-propylquinoline (0.04 g, 0.1 mmole) was dissolved in acetonitrile (20 mL). Ammonium cerium (IV) nitrate (0.055 g, 0.10 mmol) was dissolved in water (10 mL) and added to above solution. An immediate precipitate formed. Acetonitrile (20 mL) was added, along with ammonium cerium (IV) nitrate (0.005 g, 0.01 mmol). The precipitate was collected by filtration and was determined to be the product. A 20% yield was obtained. MS (ES+): m/z 302.0 M+1); ¹H NMR (CDCl₃) δ 11.9 (bs, 1H, NH), 7.90 (s, 1H, ArH), 7.69 (d, 1H, ArH), 7.45 (d, 1H, ArH), 2.81 (m, 2H, CH_{2),} 1.67 (m, 2H, CH₂), 1.2 (m, 1H, CH), 1.0 (m, 3H, CH₃), 0.65 (m, 2H, CH₂), 0.55 (m, 2H, CH₂).

### EXAMPLE 36

### 4-[(2-Cyclopropylethynyl)oxy]-6-fluoro-3-(isopropyl)-2(1H)-quinolinone

A. 6-Fluoro-3-isopropyl-4-(2,2,2-trifluoroethoxy)-2(1H)-quinolinone

The title compound was prepared from 6-fluoro-4-hydroxy-3-isopropyl-2(1H)-quinolinone by the method used in Example 35. MS (ES+): m/z 304.0 (M+1); ¹H NMR (CDCl₃) δ 12.6 (bs, 1H, NH), 7.42-7.35 (m, 2H, ArH), 7.28-7.23 (m, 1H, ArH), 4.31 (q, 2H, CH₂), 3.4 (m, 1H, CH), 1.4 (d, 6H, CH₃).

B. 2-[tert-Butyl(dimethyl)silyl]oxy-6-fluoro-3-isopropyl-4-(2,2,2-trifluoroethoxy)quinoline

6-Fluoro-3-isopropyl-4-(2,2,2-trifluoroethoxy)-2(1H)-quinolinone (0.92 g, 3.0 mmol) was dissolved in DMF (30 mL). t-Butyldimethylsilyl chloride (0.95 g, 6.0 mmol) was added followed by triethylamine (1.6 mL, 10.5 mmol). The reaction was stirred at rt for 2.5 h. It The mixture was poured into ice water (250 mL). The product was extracted with ether. The solution was dried with magnesium sulfate, filtered, and the solvent removed in vacuo to give the product in 80% yield. MS (CI+): m/z 418.0 (M+1); ¹H NMR (CDCl₃) δ 7.70 (dd, 1H, ArH), 7.48 (dd, 1H, ArH), 7.32 (dd, 1H, ArH), 4.33 (q, 2H, CH₂), 3.55 (m, 1H, CH), 1.4 (d, 6H, CH₃), 1.06 (s, 9H, Si-t-Bu), 0.43 (s, 6H, Si-CH₃).

C. 2-[tert-Butyl(dimethyl)silyl]oxy-4-[(2-cyclopropylethynyl)oxy]-6-fluoro-3-isopropylquinoline

The title compound was prepared from 2-[tert-butyl(dimethyl)silyl]oxy-6-fluoro-3-isopropyl-4-(2,2,2-trifluoroethoxy)quinoline by the method used in Example 35. GCMS (CI+): m/z 400.0 (M+1).

D. 4-[(2-Cyclopropylethynyl)oxy]-6-fluoro-3-(isopropyl)-2(1H)-quinolinone

2-[tert-Butyl(dimethyl)silyl]oxy-6-fluoro-4-[(2-cyclopropylethynyl)oxy]-3-isopropylquinoline (0.30 g, 0.75 mmol) was dissolved in ethyl acetate/acetontrile (10 mL, 1:1, v/v). t-Butylammonium fluoride hydrate (0.20 g, 1.1 mmol) was added. The reaction was stirred at rt for 5 min. Saturated sodium chloride solution was added and the product extracted with ethyl acetate. The product was isolated by chromatography on silica gel (4×7 cm column) eluted with ethyl acetate/hexane (1:2, v/v). Final purification was accomplished by chromatography on C18 (Water's Symmetry C18 column, 19×150 mm) eluted with methanol/water (3:2, v/v) to give a 20% yield. MS (ES+): m/z 286.0 (M+1); ¹H NMR (CDCl₃) δ 11.6 (bs, 1H, NH), 7.61 (d, 1H, ArH), 7.58-7.22 (m, 2H, ArH), 3.60 (m, 1H, CH), 1.4 (d, 6H, CH₃), 1.15 (m, 1H, CH), 0.68 (m, 2H, CH₂), 0.56 (m, 2H, CH₂).

### EXAMPLE 37

### 4-[(2-Cyclopropylethynyl)oxy]-6-fluoro-3-propyl-2(1H)-quinolinone

The title compound was prepared from 6-fluoro-4-hydroxy-3-propyl-2(1H)-quinolinone as in Example 36. MS (ES+): m/z 286.0 (M+1); ¹H NMR (CDCl₃) δ 10.9 (bs, 1H, NH), 7.59 (d, 1H, ArH), 7.25(m, 2H, ArH), 2,78 (m, 2H, CH₂), 1.66(m, 2H, CH₂), 1.19 (m, 1H, CH), 1.04 (t, 3H, CH₃), 0.68 (m, 2H, CH₂), 0.56 (m, 2H, CH₂).

### EXAMPLE 38

### 6-Fluoro-3-isopropyl-4-[(3-methyl-1-pentynyl)oxy]-2(1H)-quinolinone

The title compound was prepared from 6-fluoro-4-hydroxy-3-isopropyl-2(1H)-quinolinone by the sequence of step in Example 36 except sec-butyl lithium was used in step C. MS (ES+): m/z 302.0 (M+1); ¹H NMR (CDCl₃) δ 12.1 (bs, 1H, NH), 7.68 (d, 1H, ArH), 7.38-7.26 (m, 2H, ArH), 3.68 (m, 1H, CH), 2.33 (m, 1H), 1.4 (m, 8H), 1.12 (m, 3H, CH₃), 0.92(m, 3H, CH₃).

### EXAMPLE 39

### 4-(Cyclobutylmethoxy)-3-ethyl-6-fluoro-2(1H)-quinolinone

A. 3-Ethyl-6-fluoro-4-hydroxy-2(1H)-quinolinone

To a round bottom flask equipped with a stir bar, an addition funnel and nitrogen on demand was added diethyl ethyl malonate (5.0 mL, 5.0 g, 27 mmol). A solution of potassium hydroxide (1.8 g, 32 mmol) in absolute ethanol (50 mL) was added in a dropwise manner via addition funnel and the reaction was allowed to stir at rt for 18 h. The reaction mixture was concentrated under reduced pressure, the crude solid washed with diethyl ether (30 mL), and concentrated again under reduced pressure to afford potassium 2-(ethoxycarbonyl)butanoate (1.0 g, 95%) as a white solid. The product was used in the next step without further purification. To potassium 2-(ethoxycarbonyl)butanoate (1.0 g, 5.0 mmol) in a round bottom flask equipped with a stir bar and nitrogen on demand, was added methylene chloride (15 mL). Oxalyl chloride (0.44 mL, 0.64 g, 5.0 mmol) was added dropwise via syringe, followed by addition of catalytic N,N-dimnethylformamide (1 drop) via pipet. The reaction mixture was allowed to stir at rt for 18 h. When judged to be complete, the mixture was concentrated under reduced pressure to provide ethyl 2-(chlorocarbonyl)butanoate (0.95 g, >100%) as an orange oil. The product was used in the next step without further purification. In a round bottom flask equipped with a stir bar, an addition funnel, and nitrogen on demand, 4-fluoroaniline (1.9 mL, 2.2 g, 20 mmol) was dissolved in methylene chloride (100 mL) and triethylamine (10 mL, 7.3 g, 73 mmol) was added. Ethyl 2-(chlorocarbonyl)butanoate (5.1 g, 29 mmol) in methylene chloride (50 mL) was added dropwise via addition funnel and the mixture was allowed to stir at RT. When judged to be complete, the reaction mixture was partitioned between ethyl acetate and water. The organic layer was separated, dried over magnesium sulfate, filtered and concentrated under reduced pressure to afford ethyl 2-[(4-fluoroanilino)carbonyl]butanoate (4.0 g, 80%) as a crystalline solid. The product was used in the next step without further purification.

In a round bottom flask equipped with a stir bar and nitrogen on demand, 3 ethyl 2-[(4-fluoroanilino)carbonyl]butanoate (3.8 g, 15 mmol) was dissolved in PPMA (70 mL, Heterocycles, Vol. 43, No.1, 1996) and heated to 170° C. for 1 h. The mixture was allowed to cool to rt, then poured over ice and filtered. The filtrate was extracted with ethyl acetate, dried over magnesium sulfate, filtered, and concentrated under reduced pressure to a yellow solid. The crude product was dissolved in 0.5 N sodium hydroxide, extracted with toluene, and acidified to pH˜3 using concentrated hydrochloric acid. The resulting white precipitate was filtered and dried to provide 3-ethyl-6-fluoro-4-hydroxy-2(1H)-quinolinone (1.3 g, 43%) as a white solid. ¹H NMR (300 MHz, DMSO-d₆) δ 11.37 (s, 1H), 7.62 (d, 1H), 7.32 (m, 2H), 2.58 (q, 2H), 1.02 (t, 3H).

B. 4-(Cyclobutylmethoxy)-3-ethyl-6-fluoro-2(1H)-quinolinone

To sodium hydride (35 mg of 60% dispersion in oil, 0.87 mmol) in a round bottom flask equipped with a stir bar and nitrogen on demand, was added anhydrous N,N-dimethylformamide (5 mL). A solution of 3-ethyl-6-fluoro-4-hydroxy-2(1H)-quinolinone (0.15 g, 0.72 mmol) in anhydrous N,N-dimethylformamide (1 mL) was added dropwise, followed by dropwise addition of (bromomethyl)cyclobutane (0.10 g, 0.07 mL, 0.65 mmol) and the reaction was allowed to stir at rt for 48 h. The reaction mixture was quenched by dropwise addition of water and extracted with ethyl acetate. The organic layer was separated, dried over magnesium sulfate, filtered, and concentrated under reduced pressure to afford a yellow oil. The crude product was filtered through a pad of silica gel, eluding with methylene chloride and the filtrate was concentrated in vacuo. The solid was rinsed with diethyl ether, and filtered to provide the product (20 mg, 10%) as a white solid. MS (ES) 274 (M−H)⁻. ¹H NMR (300 MHz, DMSO-d₆) δ 11.77 (s, 1H), 7.37 (m, 3H), 4.00 (d, 2H), 2.88 (m, 1H), 2.58 (q, 2H), 2.15 (m, 2H), 1.95 (m, 4H), 1.02 (t, 3H).

### EXAMPLE 40

### 4-Cyclobutylmethoxy-3-isopropyl[1,6]naphthyridin-2(1H)-one

A. Ethyl 4-[(3-methylbutanoyl)amino]nicotinate

Ethyl 4-aminonicotinate, which can be prepared by the method of Ismail and Wibberley, JCS, 1967, p 2613, (0.42g, 2.5 mmol) was dissolved in TBF (Aldrich, Sure Seal, 5 mL) and ether (2 mL). A nitrogen atmosphere was provided. Triethylamine (0.35 mL, 2.5 mmol) was added and the reaction chilled to 0° C. Isovaleryl chloride (0.31 mL, 2.5 mmol) was added. The reaction was stirred for 4 h. Water (10 mL) was added and the product extracted with chloroform (3×). The combined organic fractions were back washed with water (1×) and brine (1×) and dried with MgSO₄. The solvent was removed invacuo and the product used without further purification. A 99% yield was obtained. MS (ES+): m/z 251.0 (M+1); ¹H NMR (DMSO-d₆) δ 10.6 (bs, 1H, NH), 9.0 (s, 1H, ArH), 8.6 (d, 1H, ArH), 8.35 (d, 1H, ArH), 4.4 (q, 2H, OCH₂), 2.20 (d, 2H, CH₂), 2.1 (m, 1H, CH), 1.4 (t, 2H, CH₃), 0.99 (d, 6H, CH₃).

B. 4-Hydroxy-3-isopropyl[1,6]naphthyridin-2(1H)-one

Ethyl 4-[(3-methylbutanoyl)amino]nicotinate (0.050 g, 0.19 mmol) was dissolved in THF (2 mL) and chilled to −78° C. Potassium bis(trimethylsilyl)amide (1.1 mL, 0.5M solution in toluene, 0.57 mmol) was added dropwise over several minutes. The reaction was placed in an ice bath and allowed to warm to rt overnight. The reaction was poured into ice water (10 mL) and extracted with ethyl acetate (2×). The pH of the aqueous phase and adjusted to 3 by the addition of 1N HCL solution. The product precipitated and was collected by filtration to give a 73% yield. MS (ES+): m/z 223.0 (M+1); ¹H NMR (DMSO-d₆) δ 12.4 (bs, 1H, NH), 9.26 (s, 1H, ArH), 8.6 (d, 1H, ArH), 7.50 (d, 1H, ArH), 3.3(m, overlapping with DOH, CH), 1.29 (d, 6H, CH₃).

C. 4-Cyclobutylmethoxy-3-isopropyl[1,6]naphthyridin-2(1H)-one

The title compound was prepared from 4-hydroxy-3-isopropyl[1,6]naphthyridin-2(1H)-one by the method outlined in Example 4. MS (ES+): m/z 273.0 (M+1); ¹H NMR (CDCl₃) δ 10.7 (bs, 1H, NH), 8.98 (s, 1H, ArH), 8.51 (d, 1H, ArH), 7.26 (d, 1H, ArH), 4.01 (d, 2H, OCH₂), 3.46 (m, 1H, CH), 2.91 (m, 1H, CH), 2.22 (m, 2H, alkyls), 1.99 (m, 4H, alkyls), 1.45 (d, 6H, CH₃).

### EXAMPLE 41

### 3-sec-Butyl-4-cyclobutylmethoxy[1,6]naphthyridin-2(1H)-one

A. Ethyl 4-[(3-methylpentanoyl)amino]nicotinate was prepared from ethyl 4-aminonicotinate and 3-methylpentanoyl chloride using the method outlined in Example 40. MS (ES+): m/z 265.0 (M+1); ¹H NMR (CDCl₃) δ 11.2 (bs, 1H, NH), 9.14 (s, 1H, ArH), 8.57 (d, 1H, ArH), 8.65 (d, 1H, ArH), 4.4 (m, 2H), 2.44 (m, 2H), 2.2 (m, 2H), 2.05-1.8 (m, 2H), 1.4 (m, 4H), 1.2 (m, 2H), 1-0.94 (t, 4H).

B. 3-sec-Butyl-4-hydroxy-[1,6]naphthyridin-2(1H)-one was prepared from ethyl 4-[(3-methylpentanoyl)amino]nicotinate using the method outlined in Example 40. MS (ES+): m/z 219.0 (M+1); ¹H NMR (CDCl₃) δ 12.3 (bs, 1H, NH), 9.23 (s, 1H, ArH), 8.52 (d, 1H, ArH), 7.45 (d, 1H, ArH), 3.1 (m, 1H), 1.8 (m, 1H), 1.6 (m, 1H), 1.2 (m, 3H), 0.75 (m, 3H).

C. 3-sec-Butyl-4-cyclobutylmethoxy[1,6]naphthyridin-2(1H)-one

The title compound was prepared from 3-sec-butyl-4-hydroxy-[l,6]naphthyridin-2(1H)-one by the method outlined in Example 40. MS (ES+): m/z 287.0 (M+1); ¹H NMR (CDCl₃) δ 10.7 (bs, 1H, NH), 8.98 (s, 1H, ArH), 8.51 (d, 1H, ArH), 7.26 (d, 1H, Arm), 4.00 (d, 2H, OCH₂), 3.15 (m, 1H, CH), 2.88 (m, 1H, CH), 2.2-0.8 (m, alkyls).

### EXAMPLE 42

6-Fluoro-3-isopropyl-4-[(3-methyl-2-butenyl)oxy]-2(1H)-quinolinone

The title compound was prepared from 6-fluoro-4-hydroxy-3-isopropyl-2(1H)-quinolinone and 4-bromo-2-methyl-2-butene by the method outlined in Example 2 except the reaction was complete after stirring at rt for 1 h. MS (ES+): m/z 290.0(M+1); ¹H NMR (DMSO-d₆) δ 11.6 (bs, 1H, NH), 7.34-7.29 (m, 3H, ArH), 5.56 (t, 1H, CH), 4.42 (d, 2H, OCH₂), 1.73 (s, 3H, CH₃), 1.60 (s, 3H, CH₃), 1.25 (d, 6H, CH₃).

### EXAMPLE 43

### 6-Fluoro-3-isopropyl-4-[(2-methyl-2-propenyl)oxy]-2(1H)-quinolinone

The title compound was prepared from 6-fluoro-4-hydroxy-3-isopropyl-2(1H)-quinolinone and 3-bromo-2-methyl-2-propene by the method outlined in example 2 except the reaction was finished after heating in a 120° C. oil bath for 1h. MS (ES+): m/z 276.0 (M+1); ¹H NMR (DMSO-d₆) δ 11.7 (bs, 1H, NH), 7.40-7.17 (m, 3H, ArH), 5.24 (s, 1H, CH), 5.08 (s, 1H, CH), 4.42 (s, 2H, OCH₂), 3.47 (m, 1H, CH), 1.92 (s, 3H, CH₃), 1.43 (d, 6H, CH₃).

### EXAMPLE 44

### 4-(Cyclobutyhnethoxy)-6-fluoro-5-nitro-3-propyl-2(1H)-quinolinone

A. Ethyl 2-[(4-fluoro-3-nitroanilino)carbonyl]pentanoate

A mixture of 4-fluoro-5-nitroaniline (1.93 g, 12.4 mmol) and diethyl propylmalonate (5.0 g, 25 mmol) of was heated to reflux for 4 h. The resultant mixture was cooled and passed through a column of silica gel with methylene chloride as the eluent. The product, 1.5 g (39%), was collected as a yellow solid. ¹H NMR (DMSO-d₆) δ 10.7 (bs, 1H), 8.47-8.5 (m, 1H), 7.81-7.85 (m, 1H), 7.53 (t, 1H), 4.0-4.2 (m, 2H), 3.45 (t, 1H), 1.7-1.8 (m, 2H). 1.2-1.4 (m, 2H), 1.14 (t, 3H), 0.86 (t, 3H).

B. 6-Fluoro 4-hydroxy-5-nitro-3-propyl-2(1H)-quinolinone

A mixture of ethyl 2-[(4-fluoro-3-nitroanilino)carbonyl]pentanoate (1.5 g, 4.8 mmol) and sodium carbonate (1.0 g, 9.6 mmol) of in water (20 mL) was heated to reflux for 1 h. The reaction mixture was cooled and acidified with 2N HCl. A precipitate formed and was collected by filtration and washed repeatedly with water. After drying, 1.24 g of a yellow solid was obtained. The solid was mixed with polyphosphoric acid (20 mL) and was heated to 120° C. for 4 h. The reaction mixture was cooled, 2N HCl was added (20 mL). A precipitate formed and was collected and dried. The product (0.94 g) was obtained as a light yellow solid in 74% yield. ¹H NMR (DMSO-d₆) δ 11.8 (s, 1H), 11.0 (br s, 1H), 7.72 (t, 1H), 7.48 (dd, 1H), 2.5 (m, overlapping with DMSO), 1.4-1.5 (m, 2H), 0.92 (t, 3H).

### 4-(Cyclobutylmethoxy)-6-fluoro-5-nitro-3-propyl-2(1H)-quinolinone

To a stirred suspension of sodium hydride (0.25 g of a 50% dispersion in oil, 5.3 mmol) in DMF (10 mL) was added portionwise 0.94 g of 6-fluoro 4-hydroxy-5-nitro-3-propyl-2(1H)-quinolinone. After stirring for 10 min, cyclobutylethyl-bromide (0.47 mL, 4.2 mmol) was added. The reaction mixture was stirred for 24 h. The mixture was then concentrated and dry-packed on silica gel. Flash column chromatography on silica with 5% methanol in methylene chloride gave 0.16 g (14%) of the product as a peach-colored solid. ¹H NMR (DMSO-d₆) δ 12.23 (br s, 1H), 7.75 (t, 1H), 7.54 (dd, 1H), 3.8 (d, 2H), 2.6-2.8 (m, 1H), 2.5 (m, overlapping with DMSO), 1.8-2.2 (m, 6H), 1.5-1.7 (m, 2H), 0.95 (t, 3H).

### EXAMPLE 45

### 4-[(2-Cyclopropylethynyl)oxy]-3-ethyl-6-fluoro-2(1H)-quinolinone

In a round bottom flask equipped with a stir bar, and nitrogen on demand, 2-{[tert-Butyl(dimethyl)silyl]oxy}-3-ethyl-6-fluoro-4-(2,2,2-trifluoroethoxy) quinoline (0.2 g, 0.5 mmol) (prepared from 3-ethyl-6-fluoro-4-hydroxy-2(1H)-quinolinone as in Example 36) was dissolved in anhydrous diethyl ether (6 mL) and cooled to −78° C. by means of a dry ice/acetone bath. Cyclopropyl lithium (3.5 mL of a 0.50M solution in ether, 1.76 mmol) was added dropwise and the mixture was allowed to stir at −78° C. for 1h and was then allowed to warm to rt. When judged to be complete, the reaction mixture was quenched with saturated ammonium chloride and the layers were separated. The organic layer was dried over sodium sulfate, filtered and concentrated under reduced pressure. The remaining residue was dissolved in THF and acidic resin (BioRad, AG 50W-X4, 200-400 mesh, hydrogen form) was added to the mixture. When the deprotection was judged to be complete, the reaction was filtered and concentrated in vacuo. The crude material was purified by column chromatography, eluding with 3:1 hexanes/ethyl acetate to afford the product, 18 mg, 10% yield, as a white solid. MS (ES) 270 (M−H)⁻. ¹H NMR (DMSO-d₆) δ 12.11 (s, 1H), 7.41 (m, 3H), 2.68 (q, 2H), 1.25 (m, 1H), 1.15 (t, 3H), 0.76 (m, 2H), 0.55 (m, 2H).

### EXAMPLE 46

### 6-Chloro-4-[(2-Cyclopropylethynyl)oxy]-3-isopropyl-2(1H)-quinolinone

2-[tert-Butyl(dimethyl)silyl]oxy-6-chloro-3-isopropyl-4-(2,2,2-trifluoroethoxy)quinoline (108 mg, 0.25 mmol) was prepared from 6-chloro-3-isopropyl-2(1H)-quinolinone (Example 9 step A )and treated as previously described in Example 36 steps B, C, and D. to give the crude product which was purified by flash chromatography (20% ethyl acetate/hexanes) to give the title compound (10 mg, 9%) as a white solid. MS (ES−) 300 (M−H)⁻. ¹H NMR (CDCl₃, 400 MHz) δ 12.47 (s, 1M), 7.92 (d, 1H), 7.47 (m, 1H), 7.33 (d, 1H), 3.64 (m, 1H), 1.45 (d, 6H), 1.18 (m, 1H), 0.71 (m, 2H), 0.59 (m, 2H).

### EXAMPLE 47

### 4-[(2-Cyclopropylethynyl)oxy]-6-fluoro-3-isobutyl-2(1,H)-quinolinone

A. 6-Fluoro-4-hydroxy-3-isobutyl-2(1H)-quinolone

The title compound was prepared according to the method of Cai et al. (J. Med. Chem. 1996, 39, 3248-3255): 4-fluoroaniline (15.87 g, 143 mmol) and diethyl isobutyl malonate (71.06 g, 329 mmol) were heated to 180° C. The ethanol produced was collected in a Dean-Stark trap (5ml). After 6 h, the solution was cooled to room temperature and a precipitate formed. The mixture was combined with methanol (80 ml), water (400 ml), and sodium carbonate (52 g) and heated to reflux for 1 h. Incomplete hydrolysis was observed. The mix was neutralized with 2N HCl and filtered. The resulting solid was treated with 2N LiOH (200 ml) and THF (200 ml) with stirring for 48 h. The THF was removed in vacuo and a precipitate was observed. The solid was filtered and the filtrate was acidified to pH 1 with conc. HCl, and the resulting solid filtered. The solid was treated with polyphosphoric acid (300 ml) at 140° C. for 3 h, then cooled to room temperature. Aqueous HCl (1N, 400 ml) was added and stirred for 4 h.The pH was adjusted to 4 with 20% NaOH. The resulting solid was filtered, dried in vacuo overnight to give the title compound (21 g, 62%). MS (ES−) 234 (M−H)⁻. ¹H NMR (DMSO-d₆, 400 MHz) δ 11.30 (s, 1H), 7.58 (q, 1H1), 7.30 (m, 1H), 7.22(m, 1H), 2.43 (d, 2H), 1.87 (m, 1H), 0.83 (d, 6H).

B. 6-Chloro-3-isobutyl-4-(2,2,2-trifluoroethyl)-2(1H)-quinolone

6-Chloro-4-hydroxy-3-isobutyl-2(1H)-quinolone (5 g, 21 mmol) was alkylated as described in example 36 step A to give the title compound (634 mg, 9.5%) as a white powder. MS (ES+) 318 (M+H)⁺. ¹H N (CDCl₃, 400 MHz) 8 7.30 (m, 3H), 4.36 (q, 2H1), 2.61 (d, 2H), 2.13 (m, 1H), 0.97 (d, 6H).

C. 4-[(2-Cyclopropylethynyl)oxy]-6-fluoro-3-isobutyl-2(1H)-quinolinone

6-Chloro-3-isobutyl-4-(2,2,2-trifluoroethyl)-2(1H)-quinolone was protected as described in example 36 step B, to give 2-[tert-Butyl(dimethyl)silyl]oxy-6-fluoro-4-(2,2,2-trifluoroethyl)-3-isobutylquinolinone. The product was purified by flash chromatography (2% ethyl acetate/hexanes, neutral alumina) to give an oil which was not characterized. The product was treated as described in example 36 step C to prepare the silyl-protected cyclopropyl-alkynyl ether. Following deprotection, see example 45, the crude product was obtained. The product was purified by flash chromatography (20% ethyl acetate/hexane) to give the title compound (17 mg, 10%) as a white solid. MS (ES+) 300 (M+H)⁺. ¹H NMR (CDCl₃, 400 MHz) δ 12.45 (s, 1H), 7.65 (dd, 1H), 7.40 (dd, 1H), 7.29 (m, 1H), 2.76 (d, 2H), 2.13 (quint, 1H), 1.18 (m, 1H), 1.00 (d, 6H), 0.71 (m, 2H), 0.59 (m, 2H).

### EXAMPLE 48

### 4-{[(E)-2-Cyclopropylethenyl]oxy}-6-fluoro-3-isopropyl-3,4-dihydro-2(1H)-quinolinone

2-[tert-Butyl(dimethyl)silyl]oxy-4-[(2-cyclopropylethynyl)oxy]-6-fluoro-3-isopropylquinoline (0.1 g, 0.25 mmoles, see Example 36 step C) was dissolved in THF (2 mLs, Aldrich Sureseal). Lithium aluminum hydride (0.375 μL, 0.375 mmoles, 1.5 eq, 1M solution in THF) was added at rt. After 30 minutes, 0.25 mL of water was added to quench the reaction. The mixture was filtered and the solvents removed in vacuo. The product was isolated by chromatography on neutral alumina eluted with ethyl acetate/hexane (5:95, v/v). The silyl-protecting group was removed as in example 45. The product was purified by chromatography on silica gel eluted with ethyl acetate/hexane (1:4, v/v). The product was obtained in 20% yield. MS (ES+): m/z 288.0 (M+1); ¹H NMR (CDCl₃) δ 12.8 (bs, 1H, NH, 7.4 (m, 2H, ArH), 7. 2 (m, 1H, ArH), 6.5 (d, 1H, alkene-H), 4.7 (dd, 1H, alkene-H), 3.4 (m, 1H, CH), 1.4 (d, 6H, CH₃), 1.15 (m, 1H, CH), 0.68 (m, 2H, CH₂), 0.56 (m, 2H, CH₂).

### EXAMPLE 49

### 4-Cyclobutylmethoxy-6-fluoro-3-methyl-2(1H)-quinolinone

A. 6-Fluoro-4-hydroxy-3-methyl-2(1H)-quinolinone was prepared according to the procedure used in example 47 starting from 4-fluoroaniline (71.6 mmol, 7.95 g) and methyl diethyl malonate (164 mmol, 28.54 g) to give 9.8 g of product (71%) as a white solid: MS (ES+): m/z 194, (+1), ¹H NMR (d₆DMSO) δ 11.38 (s, 1H), 10.19 (s, 1H), 7.55 (dd, 1H, J=2.9, 9.9 Hz), 7.39-7.22 (m, 2H), 2.04 (d, .5H, J=1.8 Hz), 1.96 (s, 2.5H).

B. 4-Cyclobutylmethoxy-6-fluoro-3-methyl-2(1fl)-quinolinone was prepared from 6-fluoro-4-hydroxy-3-methyl-2(1H)-quinolinone by the method used in example 2 except the oil bath temperature was 70° C. and the reaction time was 24 hrs. The product was isolated by chromatography on silica gel eluted with CHCl₃/MeOH (95/5). A 9% yield was obtained. MS (ES+): m/z 262.0 (M+1); ¹H NMR (DMSO-d₆) δ 12.3 (bs, 1H, NH), 7.44-7.41 (m, 2H, ArH), 7.24-7.19 (m, 1H, ArH), 4.01 (d, 2H, OCH₂), 2.9 (m, 1H, CH), 2.2 (m, 5H, alkyls), 2.0 (m, 4H, alkyls).

### EXAMPLE 50

### Anti-HIV Activity

The compounds of the present invention were tested for anti-HIV activity in MT₄ cells according to the method described by Averett, D. R., J. Virol. Methods, 23, 1989, 263-276 and was found to have an IC₅₀ in the range of 0.005-2 μM (Table 1).

TABLE I

| | Activity |
|---|---|
| Example Number | Range |
| 1: 6-Chloro-4-(cyclohexyloxy)-3-propyl-2(1H)-quinolinone | A |
| 9: 6-Chloro-4-(cyclohexyloxy)-3-isopropyl-2(1H)-quinolinone | A |
| 20: 6-Chloro-4-(cyclohexyloxy)-3-propyl-2(1H)-quinolinethione | A |
| 35: 6-Chloro-4-[(2-cyclopropylethynyl)oxy]-3-propyl-2(1H)- | A |
| quinolinone | |
| 37: 4-[(2-Cyclopropylethynyl)oxy]-6-fluoro-3-propyl-2(1H)- | A |
| quinolinone | |
| 45: 4-[(2-Cyclopropylethynyl)oxy]-3-ethyl-6-fluoro-2(1H)- | A |
| quinolinone | |
| 46: 6-Chloro-4-[(2-Cyclopropylethynyl)oxy]-3-isopropyl-2(1H)- | A |
| quinolinone | |
| 47: 4-[(2-Cyclopropylethynyl)oxy]-6-fluoro-3-isobutyl-2(1H)- | A |
| quinolinone | |
| 36: 4-[(2-Cyclopropylethynyl)oxy]-6-fluoro-3-isopropyl-2(1H)- | A |
| quinolinone | |
| 24: 6-Chloro-4-(cyclohexylsulfinyl)-3-propyl-2(1H)-quinolinone | B |
| 3: 6-Chloro-4-(cyclopropylmethoxy)-3-propyl-2(1H)- | B |
| quinolinone | |
| 10: 4-Cyclopentyloxy-6-methyl-3-propyl-2(1H)-quinolinone | C |
| 5: 6-Chloro-4-(cyclohexylmethoxy)-3-propyl-2(1H)- | C |
| quinolinone | |
| 6: 6-Chloro-4-(1,3-dioxolan-2-ylmethoxy)-3-propyl-2(1H)- | C |
| quinolinone | |

Activity ranges: A (IC₅₀=0.005-0.1 μM); B (IC₅₀32 0.1-0.5 μM), C (IC₅₀=0.5-2 μM).

### EXAMPLE 51

### Tablet Formulation

The following formulations A, B and C are prepared by wet granulation of the ingredients with a solution of povidone, followed by addition of magnesium stearate and compression.

| | mg/tablet |
|---|---|
| Formulation A | |
| Active Ingredient | 250 |
| Lactose B.P. | 210 |
| Povidone B.P. | 15 |
| Sodium Starch Glycollate | 20 |
| Magnesium Stearate | 5 |
| | 500 |
| Formulation B | |
| Active Ingredient | 250 |
| Lactose B.P. | 150 |
| Avicel PH 101 | 60 |
| Povidone B.P. | 15 |
| Sodium Starch Glycollate | 20 |
| Magnesium Stearate | 5 |
| | 500 |
| Formulation C | |
| Active Ingredient | 250 |
| Lactose B.P. | 200 |
| Starch | 50 |
| Povidone | 5 |
| Magnesium Stearate | 4 |
| | 359 |

The following formulations, D and E, are prepared by direct compression of the admixed ingredients. The lactose in formulation E is of the direct compression type (Dairy Crest-“Zeparox”).

| | mg/tablet |
|---|---|
| Formulation D | |
| Active Ingredient | 250 |
| Pregelatinized Starch NF15 | 150 |
| | 400 |
| Formulation E | |
| Active Ingredient | 250 |
| Lactose B.P. | 150 |
| Avicel | 100 |
| | 500 |

Formulation F (Controlled Release Formulation)

The formulation is prepared by wet granulation of the ingredients with a solution of povidone followed by the addition of magnesium stearate and compression.

| | mg/tablet |
|---|---|
| Active Ingredient | 500 |
| Hydroxypropylmethylcellulose | 112 |
| (Methocel K4M Premium) | |
| Lactose B.P. | 53 |
| Povidone B.P. | 28 |
| Magnesium Stearate | 7 |
| | 700 |

Drug release takes place over a period of about 6-8 hours and is complete after 12 hours.

### EXAMPLE 52

### Capsule Formulations

Formulation A

A capsule formulation is prepared by admixing the ingredients of formulation D in Example 51 above and filling into a two-part hard gelatin capsule. Formulation B (infra) is prepared in a similar manner.

| | mg/capsule |
|---|---|
| Formulation B | |
| Active Ingredient | 250 |
| Lactose B.P. | 143 |
| Sodium Starch Glycollate | 25 |
| Magnesium Stearate | 2 |
| | 420 |
| Formulation C | |
| Active Ingredient | 250 |
| Macrogel 4000 B.P. | 350 |
| | 600 |

Capsules of formulation C are prepared by melting the Macrogel 4000 B.P., dispersing the active ingredient in the melt and filling the melt into a two-part hard gelatin capsule.

| Formulation D | mg/capsule |
|---|---|
| Active Ingredient | 250 |
| Lecithin | 100 |
| Arachis Oil | 100 |
| | 450 |

Capsules of formulation D are prepared by dispersing the active ingredient in the lecithin and arachis oil and filling the dispersion into soft, elastic gelatin capsules.

| Formulation E | mg/capsule |
|---|---|
| Active Ingredient | 150.0 |
| Vitamin E TPGS | 400.0 |
| Polyethylene Glycol 400 NF | 200.5 |
| Propylene Glycol USP | 39.5 |

Four (4) kilograms (kg) of Vitamin E TPGS (obtained from Eastman Chemical Co.) was heated at 50° C. until liquefied. To the liquified Vitamin E TPGS, 2.005 kg of polyethylene glycol 400 (PEG400) (low aldehyde, <10 ppm, obtained from Union Carbide or Dow Chemical Co.) heated to 50° C. was added and mixed until a homogeneous solution was formed. The resultant solution was heated to 65° C. 1.5 kg of active ingredient was dissolved in the liquefied solution of Vitamin E TPGS and PEG 400. 0.395 kg of propylene glycol at room temperature was added and mixed until a homogenous solution was formed. The solution was cooled to 28-35° C. The solution was then de-gassed. The mixture was preferably encapsulated at 28-35° C. at a fill weight equivalent to 150 mg of volatiles-free compound, into Size 12 oblong, white opaque soft gelatin capsules using a capsule filling machine. The capsule shells were dried to a constant fill moisture of 3-6% water and a shell hardness of 7-10 Newtons, and placed in a suitable container.

Formulation F (Controlled Release Capsule)

The following controlled release capsule formulation is prepared by extruding ingredients a, b, and c using an extruder, followed by spheronization of the extrudate and drying. The dried pellets are then coated with release-controlling membrane (d) and filled into a two-piece, hard gelatin capsule.

| | | mg/capsule |
|---|---|---|
| (a) | Active Ingredient | 250 |
| (b) | Microcrystalline Cellulose | 125 |
| (c) | Lactose B.P. | 125 |
| (d) | Ethyl Cellulose | 13 |
| | | 513 |

### EXAMPLE 53

### Injectable Formulation

| Formulation A | mg |
|---|---|
| Active Ingredient | 200 |
| Hydrochloric Acid Solution 0.1 M or | |
| Sodium Hydroxide Solution 0.1 M q.s. to pH | 4.0 to 7.0 |
| Sterile water q.s. to | 10 ml |

The active ingredient is dissolved in most of the water (35°-40° C.) and the pH adjusted to between 4.0 and 7.0 with the hydrochloric acid or the sodium hydroxide as appropriate. The batch is then made up to volume with water and filtered through a sterile micropore filter into a sterile 10 ml amber glass vial (type 1) and sealed with sterile closures and overseals.

| Formulation B | |
|---|---|
| Active Ingredient | 125 mg |
| Sterile, Pyrogen-free, pH 7 Phosphate | 25 ml |
| Buffer, q.s. to | |

### EXAMPLE 54

### Intramuscular Injection

| | | |
|---|---|---|
| Active Ingredient | 200 | mg |
| Benzyl Alcohol | 0.10 | g |
| Glycofurol 75 | 1.45 | g |
| Water for injection q.s. to | 3.00 | ml |

The active ingredient is dissolved in the glycofarol. The benzyl alcohol is then added and dissolved, and water added to 3 ml. The mixture is then filtered through a sterile micropore filter and sealed in sterile 3 mL amber glass vials (type 1).

### EXAMPLE 55

### Syrup

| | | |
|---|---|---|
| Active Ingredient | 250 | mg |
| Sorbitol Solution | 1.50 | g |
| Glycerol | 2.00 | g |
| Sodium Benzoate | 0.005 | g |
| Flavor, Peach 17.42.3169 | 0.0125 | ml |
| Purified Water q.s. to | 5.00 | ml |

The active ingredient is dissolved in a mixture of the glycerol and most of the purified water. An aqueous solution of the sodium benzoate is then added to the solution, followed by addition of the sorbital solution and finally the flavor. The volume is made up with purified water and mixed well.

### EXAMPLE 56

### Suppository

| | mg/capsule suppository |
|---|---|
| Active Ingredient | 250 |
| Hard Fat, B.P. (Witepsol H15-Dynamit Nobel) | 1770 |
| | 2020 |

One-fifth of the Witepsol H15 is melted in a steam-jacketed pan at 45° C. maximum. The active ingredient is sifted through a 200 μm sieve and added to the molten base with mixing, using a Silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 45° C. At a temperature of 38° C. to 40° C., 2.02 g of the mixture is filled into suitable, 2 ml plastic molds. The suppositories are allowed to cool to room temperature.

| Example 57: Pessaries | mg/pessary |
|---|---|
| Active Ingredient | 250 |
| Anhydrate Dextrose | 380 |
| Potato Starch | 363 |
| Magnesium Stearate | 7 |
| | 1000 |

1. A compound of formula (Ia) 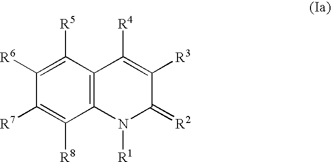
wherein:
R¹ is hydrogen;
R² is oxygen or sulfur;
R³ is trifluoromethyl; cyano; C₁₋₈alkyl optionally substituted with C₁₋₈alkyl or trifluoromethyl;
or OR¹⁵, wherein R¹⁵ is C₁₋₈alkyl optionally substituted with C₁₋₈alkyl;
R⁴ is
OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;
heterocyclealkynyl; C₃₋₆cycloalkylalkenyl; C₆₋₁₄arylalkynyl; C₃₋₆cycloalkylalkynyl;
SR¹², wherein R¹² is C₃₋₆Cycloalkyl;
S(O)R¹², wherein R¹² is C₃₋₆cycloalkyl; or
NR¹³R¹⁴ wherein R¹³ and R¹⁴, which may be the same or different, are hydrogen or C₁₋₈alkyl, optionally substituted with C₁₋₈alkyl;
R⁵ is hydrogen; nitro; halogen; C₁₋₈alkyl, optionally substituted with C₁₋₈alkyl or trifluoromethyl;
R⁶ is hydrogen; halogen; C₁₋₈alkyl; cyano; trifluoromethyl; or OR¹⁰ wherein R¹⁰ is C₁₋₈alkyl or trifluoromethyl;
R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;
R⁸ is hydrogen; halogen; cyano; nitro; or OR¹⁶, wherein R¹⁶ is hydrogen or C₁₋₈alkyl optionally substituted with C₁₋₈alkyl or trifluoromethyl;
provided that R₆ and R₇ cannot both be hydrogen; and further provided that when R¹ is H, R² is O, R³ is C₁₋₈alkyl, R⁴ is OR¹¹ wherein R¹¹ is C₁₋₈alkyl, R⁵ is H, R⁶ is H or OR¹⁰ wherein R¹⁰ is C₁₋₈alkyl, R⁷ is H, C₁₋₈alkyl, or OR⁹ wherein R⁹ is C₁₋₈alkyl, then R⁸ cannot be H or OR¹⁶ wherein R¹⁶ is H or C₁₋₈alkyl;
or a pharmaceutically acceptable derivative thereof.

2. A compound of formula (Ib) wherein: 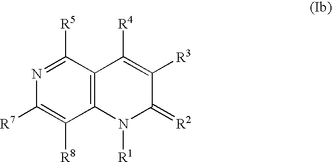
R¹ is hydrogen;
R² is oxygen or sulfur;
R³ is trifluoromethyl; cyano; C₁₋₈alkyl optionally substituted with C₁₋₈alkyl or trifluoromethyl;
or OR¹⁵, wherein R¹⁵ is C₁₋₈alkyl optionally substituted with C₁₋₈alkyl;
R⁴ is
OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;
heterocyclealkynyl; C₃₋₆cycloalkylalkenyl; C₆₋₁₄arylalkynyl; C₃₋₆cycloalkylalkynyl;
SR¹², wherein R¹² is C₃₋₆cycloalkyl;
S(O)R¹², wherein R¹² is C₃₋₆cycloalkyl; or
NR¹³R¹⁴ wherein R¹³ and R¹⁴, which may be the same or different, are hydrogen or C₁₋₈alkyl, optionally substituted with C₁₋₈alkyl;
R⁵ is hydrogen; nitro; halogen; C₁₋₈alkyl, optionally substituted with C₁₋₈alkyl or trifluoromethyl;
R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;
R⁸ is hydrogen; halogen; cyano; nitro; or OR¹⁶, wherein R¹⁶ is hydrogen or C₁₋₈alkyl optionally substituted with C₁₋₈alkyl or trifluoromethyl;
or a pharmaceutically acceptable derivative thereof.

3. A compound of formula (Ic) wherein: 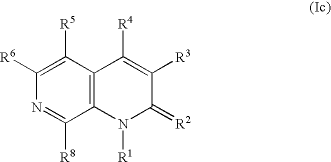
R¹ is hydrogen;
R² is oxygen or sulfur;
R³ is trifluoromethyl; cyano; C₁₋₈alkyl optionally substituted with C₁₋₈alkyl or trifluoromethyl;
or OR¹⁵, wherein R¹⁵ is C₁₋₈alkyl optionally substituted with C₁₋₈alkyl;
R⁴ is
OR¹¹, wherein R¹¹ is C₂₋₈alkenyl optionally substituted with C₁₋₈alkyl; C₁₋₈alkyl optionally substituted with C₁₋₈alkyl; C₆₋₁₄arylalkyl; C₃₋₆cycloalkyl; C₃₋₆cycloalkylalkyl; heterocyclealkyl;
heterocyclealkynyl; C₃₋₆cycloalkylalkenyl; C₆₋₁₄arylalkynyl; C₃₋₆cycloalkylalkynyl;
SR¹², wherein R¹² is C₃₋₆cycloalkyl;
S(O)R¹², wherein R¹² is C₃₋₆cycloalkyl; or
NR¹³R¹⁴ wherein R¹³ and R¹⁴, which may be the same or different, are hydrogen or C₁₋₈alkyl, optionally substituted with C₁₋₈alkyl;
R⁵ is hydrogen; nitro; halogen; C₁₋₈alkyl, optionally substituted with C₁₋₈alkyl or trifluoromethyl;
R⁶ is hydrogen; halogen; C₁₋₈alkyl; cyano; trifluoromethyl; or OR¹⁰ wherein R¹⁰ is C₁₋₈alkyl or trifluoromethyl;
R⁸ is hydrogen; halogen; cyano; nitro; or OR¹⁶, wherein R¹⁶ is hydrogen or C₁₋₈alkyl optionally substituted with C₁₋₈alkyl or trifluoromethyl;
or a pharmaceutically acceptable derivative thereof.

4. A compound of formula (Ia) according to claim 1 wherein:
R⁴ is hydrogen;
R² is oxygen;
R³ is trifluoromethyl; cyano; C₁₋₈alkyl optionally substituted with C₁₋₈alkyl or trifluoromethyl;
R⁴ is
OR¹¹, wherein R¹¹ is C₂₋₈alkenyl optionally substituted with C₁₋₈alkyl; C₆₋₁₄arylalkyl; C₃₋₆cycloalkyl; C₃₋₆cycloalkylalkyl; heterocyclealkyl; heterocyclealkynyl; C₃₋₆cycloalkylalkenyl; C₆₋₁₄arylalkynyl; C₃₋₆cycloalkylalkynyl;
SR¹², wherein R¹² is C₃₋₆cycloalkyl; or
S(O)R¹², wherein R¹² is C₃₋₆cycloalkyl;
R⁵ is hydrogen; nitro; halogen; C₁₋₈alkyl, optionally substituted with C₁₋₈alkyl or trifluoromethyl;
R⁶ is halogen; cyano; trifluoromethyl;
R⁷ is hydrogen; C₁₋₈alkyl; halogen; C₆₋₁₄aryl; C₁₋₈alkylaryl; C₂₋₈alkynyl; heteroaryl; or OR⁹ wherein R⁹ is C₁₋₈alkyl;
R⁸ is hydrogen; halogen; cyano; nitro; or OR¹⁶, wherein R¹⁶ is hydrogen or C₁₋₈alkyl optionally substituted with C₁₋₈alkyl or trifluoromethyl;
or a pharmaceutically acceptable derivative thereof.

5. A compound of formula (Ia) according to claim 1 wherein:
R¹ is hydrogen;
R² is oxygen;
R³ is C₁₋₈alkyl optionally substituted with C₁₋₈alkyl;
R⁴ is OR¹¹, wherein R¹¹ is C₁₋₈alkyl optionally substituted with C₁₋₈alkyl;
C₆₋₁₄arylalkyl; C₃₋₆cycloalkyl; C₃₋₆cycloalkylalkyl; heterocyclealkyl; C₃₋₆cycloalkylalkenyl; C₃₋₆cycloalkylalkynyl; or SR¹² wherein R¹² is C₃₋₆cycloalkyl;
R⁵, R⁷, and R⁸ are hydrogen;
R⁶ is halogen;
or a pharmaceutically acceptable derivative thereof.

6. A compound of formula (Ia) according to claim 1 wherein:
R¹ is hydrogen;
R² is oxygen;
R³ is C₁₋₈alkyl optionally substituted with C₁₋₈alkyl;
R⁴ is OR¹¹, wherein R¹¹ is C₆₋₁₄arylalkyl, C₃₋₆cycloalkyl, C₃₋₆cycloalkylalkyl, heterocyclealkyl, C₃₋₆cycloalkylalkenyl, or C₃₋₆cycloalkylalkynyl;
R⁵, R7, and R⁸ are hydrogen;
R⁶ is halogen;
or a pharmaceutically acceptable derivative thereof.

7. A compound formula (Ib) according to claim 2 wherein
R¹ is hydrogen;
R² is oxygen;
R³ is C₁₋₈alkyl optionally substituted with C₁₋₈alkyl;
R⁴ is OR¹¹, wherein R¹¹ is C₁₋₈alkyl optionally substituted with C₁₋₈alkyl;
C₆₋₁₄arylalkyl; C₃₋₆cycloalkyl; C₃₋₆-cycloalkylalkyl; heterocyclealkyl;C₃₋₆cycloalkylalkenyl;
C₃₋₆cycloalkylalkynyl; or SR¹² wherein R¹² is C₃₋₆cycloalkyl;
R⁵, R⁷, and R⁸ are hydrogen;
or a pharmaceutically acceptable derivative thereof.

8. A compound of formula (Ic) according to claim 3 wherein
R¹ is hydrogen;
R² is oxygen;
R³ is C₁₋₈alkyl optionally substituted with C₁₋₈alkyl; R⁴ is OR¹¹, wherein R¹¹ is C₁₋₈alkyl optionally substituted with C₁₋₈alkyl; C₆₋₁₄arylalkyl; C₃₋₆cycloalkyl; C₃₋₆cycloalkylalkyl; heterocyclealkyl; C₃₋₆cycloalkylalkenyl; C₃₋₆cycloalkylalkynyl; or SR¹² wherein R¹² is C₃₋₆cycloalkyl;
R⁵ is hydrogen;
R⁶ is halogen;
R⁸ is hydrogen;
or a pharmaceutically acceptable derivative thereof.

9. A compound selected from the group consisting of
6-Chloro-4-(cyclohexyloxy)-3-propyl-2(1H)-quinolinone;
6-Chloro-4-(cyclobutylmethoxy)-3-propyl-2(1H)-quinolinone;
6-Chloro-4-(cyclopropylmethoxy)-3-propyl-2(1H)-quinolinone;
6-Chloro-4-(cyclopentyloxy)-3-propyl-2(1H)-quinolinone;
6-Chloro-4-(cyclohexylmethoxy)-3-propyl-2(1H)-quinolinone;
6-Chloro-4-(1,3-dioxolan-2-ylmethoxy)-3-propyl-2(1H)-quinolinone;
4-Benzyloxy-6-chloro-3-propyl-2(1H)-quinolinone;
6-Chloro-4-(cyclobutyloxy)-3-propyl-2(1H)-quinolinone;
6-Chloro-4-(cyclohexyloxy)-3-isopropyl-2(1H)-quinolinone;
4-Cyclopentyloxy-6-methyl-3-propyl-2(1H)-quinolinone;
4-Cyclopentyloxy-6-methoxy-3-propyl-2(1H)-quinolinone;
4-(Cyclopentyloxy)-3-propyl-6-trifluoromethoxy-2(1H)-quinolinone;
4-Cyclopentyloxy-2-oxo-3-propyl-1,2-dihydro-6-quinolinecarbonitrile
6-Bromo-4-(cyclopentyloxy)-3-propyl-2(1H)-quinolinone;
4-Cyclopentyloxy-3-propyl-2(1H)-quinolinone;
6-Chloro-4-cyclobutylmethoxy-3-isopropyl-2(1H)-quinolinone;
6-Chloro-4-(cyclopentyloxy)-3-ethyl-2(1H)-quinolinone;
3-(sec-Butyl)-6-chloro-4-(cyclopentyloxy)-2(1H)-quinolinone;
6-Chloro-4-(cyclopentyloxy)-3-isobutyl-2(1H)-quinolinone;
6-Chloro-4-(cyclohexyloxy)-3-propyl-2(1H)-quinolinethione;
6-Chloro-4-(cyclobutylmethoxy)-3-propyl-2(1H)-quinolinethione;
3-(sec-Butyl)-6-chloro-4-(cyclopentyloxy)-2(1H)-quinolinethione;
6-Chloro-4-(cyclohexylsulfanyl)-3-propyl-2(1H)-quinolinone;
6-Chloro-4-(cyclohexylsulfinyl)-3-propyl-2(1H)-quinolinone;
4-Cyclopentyloxy-6-fluoro-3-propyl-2(1H)-quinolinone;
4-Cyclobutylmethoxy-6-fluoro-3-propyl-2(1H)-quinolinone;
3-sec-Butyl-4-(cyclobutyhmethoxy)-6-fluoro-2(1H)-quinolinone;
4-(Cyclobutylmethoxy)-6-fluoro-3-isopropyl-2(1H)-quinolinone;
4-Cyclopentyloxy-6-fluoro-3-propyl-2(1H)-quinolinethione;
4-(Cyclobutylmethoxy)-6-fluoro-3-propyl-2(1H)-quinolinethione;
4-(Cyclobutylmethoxy)-6-fluoro-3-isopropyl-2(1H)-quinolinethione;
6-Chloro-4-(isobutylamino)-3-propyl-2(1H)-quinolinone;
6- Chloro-4-(cyclobutylmethoxy)-3-ethoxy-2(1H)-quinolinone;
4-(Cyclobutylmethoxy)-3-ethoxy-6-fluoro-2(1H)-quinolinone;
6-Chloro-4-[(2-cyclopropylethynyl)oxy]-3-propyl-2(1H)-quinolinone;
4-[(2-Cyclopropylethynyl)oxy]-6-fluoro-3-(isopropyl)-2(1H)-quinolinone;
4-[(2-Cyclopropylethynyl)oxy]-6-fluoro-3-propyl-2(1H)-quinolinone;
6-Fluoro-3-isopropyl-4-[(3-methyl-1-pentynyl)oxy]-2(-quinolinone;
4-(Cyclobutylmethoxy)-3-ethyl-6-fluoro-2(1H)-quinolinone;
4-Cyclobutylmethoxy-3-isopropyl[1,6]naphthyridin-2(1H)-one;
3-sec-Butyl-4-cyclobutyhmethoxy[1,6]naphthyridin-2(1H)-one;
6-Fluoro-3-isopropyl-4-[(3-methyl-2-butenyl)oxy]-2(1H)-quinolinone;
6-Fluoro-3-isopropyl-4-[(2-methyl-2-propenyl)oxy]-2(1H)-quinolinone;
4-(Cyclobutylmethoxy)-6-fluoro-5-nitro-3-propyl-2(1H)-quinolinone;
4-[(2-Cyclopropylethynyl)oxy]-3-ethyl-6-fluoro-2(1H)-quinolinone;
6-Chloro-4-[(2-Cyclopropylethynyl)oxy]-3-isopropyl-2(1H)-quinolinone;
4-[(2-Cyclopropylethynyl)oxy]-6-fluoro-3-isobutyl-2(1H)-quinolinone;
4- {[(E)-2-Cyclopropylethenyl]oxy}-6-fluoro-3-isopropyl-3,4-dihydro-2(1H)-quinolinone;
4-Cyclobutylmethoxy-6-fluoro-3-methyl-2(1H)-quinolinone;
and pharmaceutically acceptable derivatives thereof.

10. A compound selected from the group consisting of
6-Chloro-4-[(2-cyclopropylethynyl)oxy]-3-propyl-2(1H)-quinolinone (example 35);
4-[(2-Cyclopropylethynyl)oxy]-6-fluoro-3-propyl-2(1H)-quinolinone (example 37);
4-[(2-Cyclopropylethynyl)oxy]-3-ethyl-6-fluoro-2(1H)-quinolinone (example 45);
6-Chloro-4-[(2-Cyclopropylethynyl)oxy]-3-isopropyl-2(1H)-quinolinone (example 46);
4-[(2-Cyclopropylethynyl)oxy]-6-fluoro-3-isobutyl-2(1H)-quinolinone (example 47);
4-[(2-Cyclopropylethynyl)oxy]-6-fluoro-3-isopropyl-2(1H)-quinolinone (example 36);
and pharmaceutically acceptable derivatives thereof.

11. A compound of formula (Ia), (Ib) or (Ic) according to any of claims 1 to 10 wherein the pharmaceutically acceptable derivative is a salt or ester.

12. A method of treating a virus infection in a human comprising administering to said human an effective anti-virus treatment amount of a compound of formula (Ia), (Ib) or (Ic) according any of claims 1 to 10 or a pharmaceutically acceptable derivative thereof.

13. The method according to claim 12 wherein the virus is Human Immunodeficiency Virus.

14. A pharmaceutical composition comprising an effective anti-viral amount of a compound of formula (Ia), (Ib) or (Ic) according to any of claims 1 to 10 or a pharmaceutically acceptable derivative thereof together with a pharmaceutically acceptable carrier therefor.

15. The pharmaceutical composition according to claim 14, further comprising an antiviral agent other than a compound of formula (Ia), (Ib) or (Ic).

16. A pharmaceutical composition according to claim 14 or 15 in the form of a tablet or capsule.

17. A pharmaceutical composition according to claim 14 or 15 in the form of a solution, suspension, or syrup.

18. A compound of formula (Ia), (Ib) or (Ic) according to any of claims 1 to 10 for use in medical therapy.

19. Use of a compound of formula (Ia), (Ib) or (Ic) according to any of claims 1 to 10 in the manufacture of a medicament for the treatment or prophylaxis of a virus infection.
